(19) United States
(12) Patent Application Publication
(10) Pub. No.: US 2012/0172413 A1
(43) Pub. Date: Jul. 5, 2012

(54) INCREASING LIFESPAN BY MODULATING CRTC EXPRESSION OR LOCALIZATION, AND METHODS OF SCREENING FOR MODULATORS OF SAME

(75) Inventors: Andrew Dillin, San Diego, CA (US); William Mair, San Diego, CA (US); Ianessa Morantte, San Diego, CA (US); Reuben Shaw, San Diego, CA (US)

(73) Assignee: THE SALK INSTITUTE FOR BIOLOGICAL STUDIES, La Jolla, CA (US)

(21) Appl. No.: 13/380,724

(22) PCT Filed: Jun. 28, 2010
(86) PCT No.: PCT/US10/40222

§ 371 Date: Mar. 19, 2012

Related U.S. Application Data
(60) Provisional application No. 61/220,981, filed on Jun. 26, 2009.
(60) Provisional application No. 61/299,812, filed on Jan. 29, 2010.

(51) Int. Cl. A01K 67/033; A61K 31/7052; C12N 15/12; G01N 33/00

(52) U.S. Cl. 514/44 A; 800/13; 800/3; 536/23.5

(57) ABSTRACT

The invention relates to the field of longevity enhancement. More particularly, the invention provides compositions and methods relating to CRTC modulation. In certain embodiments, the invention provides compositions and methods for enhancing longevity in an organism by inhibiting CRTC activity, such as, for example, inhibiting CRTC expression or cellular localization in the organism.

### CROSS-REFERENCE TO RELATED APPLICATIONS

This application claims the benefit of priority of U.S. provisional application Ser. No. 61/220,981, filed Jun. 26, 2009, and U.S. provisional application Ser. No. 61/299,812, filed Jan. 29, 2010. The foregoing applications are incorporated herein by reference in their entirety.

### FIELD OF THE INVENTION

The invention relates to the field of longevity enhancement. More particularly, the invention provides compositions and methods relating to CRTC modulation.

### BACKGROUND OF THE INVENTION

The last decade has seen a dramatic increase in the discovery of genetic, environmental and pharmacological perturbations that slow aging and decrease age-related pathology of model organisms. Although many of these interventions seem to function through non-overlapping mechanisms², one commonality is that lifespan extension is often coupled to reductions in energy requiring processes and the rate at which energy stores are depleted. Decreasing food intake, which increases longevity in species ranging from yeast to primates, also reduces growth and reproductive rates¹. Similarly, many of the genetic manipulations that extend lifespan involve disruption to key energy sensing, growth or metabolic pathways. These include mutations that reduce Insulin/Insulin like growth factor signalling (IIS) or amino acid-sensing target of rapamycin (TOR) signalling, both of which increase lifespan in the nematode worm Caenorhabditis elegans, the fruit fly Drosophila melanogaster, and mice^{3,4,5}. Furthermore, disruption of respiratory chain complex function^{53,54} and mutations that reduce an organism's ability to perceive food⁵⁵ also increase longevity.

One mechanism by which programmed transcriptional responses can be initiated by environmental change is via post-translational modification of transcription cofactors⁵⁶, yet this mode of regulation has not been explored in the context of aging. Modification of cofactors, by phosphorylation, acetylation, glycosylation or ubiquitination can alter their ability to bind transcription factors or to recruit transcriptional machinery, prompting activation/repression of downstream target genes in response to environmental cues⁵⁶.

Many interventions that extend lifespan involve perturbations to nutrient sensing that initiate changes in downstream transcriptional regulation. Varying energy intake can trigger programmed transcriptional responses through post-translational modification of transcription factors and their cofactors, resulting in activation/suppression of target genes.

Citation or identification of any document in this application is not an admission that such document is available as prior art to the present invention.

### SUMMARY OF THE INVENTION

Described herein are methods and compositions related to increasing lifespan by modulating CRTC expression or localization.

In certain embodiments, the invention provides a recombinant C. elegans that expresses a detectable marker operably linked to a CRTC protein in intestinal cells. In some embodiments, the detectable marker is visually detected. In other embodiments, the detectable marker is spectroscopically detected. In yet other embodiments, the detectable marker is a green fluorescent protein, a yellow fluorescent protein, a blue fluorescent protein, or a red fluorescent protein.

In some embodiment, the invention relates to a method of screening test compounds to determine whether such compounds affect the activity of an AMP-activated kinase or an LKB1 kinase, said method comprising determining the effect of test compound on the localization of a CRTC protein. In further embodiments, the invention provides a method of identifying a compound that affects the activity of an AMP-activated kinase or an LKB1 kinase, comprising contacting a non-human animal with a test compound; and measuring the localization of a CRTC protein in the presence and absence of the test compound in the non-human animal, wherein a test compound that modulates the localization of the CRTC protein in the non-human animal indicates a compound that affects the activity of an AMP-activated kinase or an LKB1 kinase.

In other embodiments, the invention relates to a method of increasing lifespan in an organism, comprising modulating expression of a CRTC protein. In certain embodiments, the expression of the CRTC protein is reduced. In some embodiments, the method comprises administering an inhibitor of CRTC to the organism. In certain embodiments, the inhibitor is an siRNA.

In some embodiments, the invention relates to a method of increasing lifespan in an organism, comprising modulating localization of a CRTC protein. In certain embodiments, the method comprises modulating phosphorylation of the CRTC protein. In further embodiments, the method comprises inhibiting calcineurin activity. In some embodiments, the method comprises administering an inhibitor of calcineurin to the organism. In certain embodiments, the inhibitor is an siRNA. In yet other embodiments, the method comprises enhancing AMPK activity.

In another embodiment, the invention relates to a method of increasing lifespan in an organism, comprising modulating expression of a CREB protein.

In yet other embodiments, the invention provides a composition comprising an isolated C. elegans CRTC protein. In a further embodiment, the invention provides a composition comprising isolated nucleic acid encoding a C. elegans CRTC protein. In a particular embodiment, the CRTC protein is CRTC-1.

In some embodiments, the invention provides a method of screening test compounds to determine whether such compounds affect the activity of calcineurin, said method comprising determining the effect of the test compound on the localization of a CRTC protein. In further embodiments, the invention provides a method of screening test compounds to determine whether such compounds enhance longevity, said method comprising determining the effect of the test compound on the localization of a CRTC protein. In further embodiments, the invention provides a method of identifying a compound that affects the activity of calcineurin, comprising contacting a non-human animal with a test compound; and measuring the localization of a CRTC protein in the presence and absence of the test compound in the non-human animal, wherein a test compound that modulates the localization of the CRTC protein in the non-human animal indicates a compound that affects the activity of calcineurin.

In other embodiments, the invention provides a method of screening test compounds to determine whether such compounds enhance longevity, said method comprising determining the effect of the test compound on the localization of a CRTC protein. In further embodiments, the invention provides a method of identifying a compound that modulates longevity, comprising contacting a non-human animal with a test compound; and measuring the localization of a CRTC protein in the presence and absence of the test compound in the non-human animal, wherein a test compound that modulates the localization of the CRTC protein in the non-human animal indicates a compound that modulates longevity.

It is noted that in this disclosure and particularly in the claims, terms such as “comprises”, “comprised”, “comprising” and the like can have the meaning attributed to it in U.S. patent law; e.g., they can mean “includes”, “included”, “including”, and the like; and that terms such as “consisting essentially of” and “consists essentially of” have the meaning ascribed to them in U.S. patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF THE DRAWINGS

FIG. 1 shows that CRTC-1 regulates longevity in response to changes in the environment. a. CRTC-1 is the sole C. elegans CRTC family member, with conserved CREB, calcineurin and 14-3-3 binding domains and nuclear export signals (NES). Conserved AMPK phosphorylation sites are found at serines 76 & 179. b. CRTC-1 is expressed throughout the intestine of the worm and in neurons in the head (inset) and tail. c. RNAi inhibition of crtc-1 and tax-6 increases C. elegan wild type median lifespan by 53% and 60% respectively (P<0.0001, Log rank, in each case). d. Overexpressing aak-2c (aa1-321) increases lifespan (P<0.0001, Log rank). e-g. Coexpression of NUP-160::GFP nuclear pore complex subunit (green), CRTC-1::RFP (red) and merge. e. Under well fed conditions CRTC-1::RFP is found throughout intestinal cells. O/N starvation (f) or 33° C., O/N (g) induce cytosolic translocation of CRTC-1::RFP. h. DAPI staining (far left panel, light gray), CRTC-1::RFP localization (center left panel, light gray) and merge after heat stress. DIC image shows intestinal tract. Black arrows indicate nuclei. i. DAPI staining of C. elegans intestinal nuclei. White arrow indicates nucleolus. j. Con-focal image of NUP-160::GFP (left panel, very light gray) marking nuclear membrane and CRTC-1::RFP (right panel, light gray) after 33° C., O/N showing nuclear exclusion of CRTC-1::RFP after heat stress.

FIG. 2 shows that activation of AAK-2 and reduction of tax-6 inactivate CRTC-1 a. CRTC-1::RFP (light gray) is excluded from the nucleus of intestinal cells when coexpressed with a constitutively active AMPK catalytic subunit AAK-2c::GFP (aa1-321, T181D) but localised throughout the cell in the presence of kinase dead AAK-2c::GFP (aa1-321, T181A). b. tax-6 RNAi results in nuclear exclusion of CRTC-1::RFP (light gray). c. Combined RNAi for the two C. elegans 14-3-3 proteins results in nuclear localization of CRTC-1::RFP. All the experiments in this figure were under fed conditions at 20° C. and white arrows indicate intestinal nuclei.

FIG. 3 shows that AMPK and TAX-6 regulate CRTC-1 by shared phosphorylation sites. a. Localization of CRTC-1::RFP with mutations to conserved AMPK target serines shows double mutations of S76A, S179A together are sufficient to retain CRTC-1::RFP in the nucleus. Insets are magnification of intestinal region. b. TAX-6 regulates CRTC-1 by dephosphorlyation of AMPK target serines. Each panel depicts the posterior region of the intestine with CRTC-1::RFP localization in light gray (main). Insets are CRTC-1::RFP localization (white) in one intestinal cell along with corresponding DIC imagine with nucleus marked with dashed line. CRTC-1::RFP is present in both the nucleus and cytosol under normal conditions. tax-6 RNAi results in nuclear exclusion of CRTC-1::RFP. Serine>Alanine substitutions in CRTC-1::RFP at Ser179 and Ser76 result in increased retention of CRTC-1 within the nucleus but this is reduced by tax-6 RNAi. Double S76A, S179A mutations in CRTC-1::RFP result in strong nuclear localization that is not reduced by tax-6 RNAi.

FIG. 4 shows pharmacological activation of CRTC-1. a. 120 minutes in M9 (non-food containing buffer) results in CRTC-1::RFP (white) nuclear export in a wild type background. Addition of tricaine induces rapid CRTC-1::RFP nuclear localization within 30 minutes. This effect is dependent upon TAX-6 as CRTC-1 expressed in a tax-6 (ok2065) mutant is constitutively cytosolic and does not respond to tricaine treatment. b. DIC and CRTC-1::RFP (white) after 60 minutes of tricaine treatment showing nuclear localization of CRTC-1::RFP but no effect on CRTC-1::RFP in which the TAX-6 binding site has been mutated. c. Tricaine induced nuclear localization of CRTC-1::RFP is expedited in aak-1 (tm1944); aak-2 (ok524) mutants (AMPK null), while re-location to the cytosol upon drug removal is delayed. X axis represents time in minutes post tricaine removal. d. The effect of RNAi of AMPK family kinases on CRTC-1::RFP localization in aak-1 (tm1944); aak-2 (ok524) mutants. Three panels represent 60, 120 and 180 min after tricaine removal. Relocation of CRTC-1::RFP to the cytosol in aak-1 (tm1944); aak-2 (ok524) mutants is delayed by RNAi of MARK-3 (par-1).

FIG. 5 shows that calcineurin regulated longevity functions via CRTC-1. a. RNAi for crtc-1 or tax-6 has no effect in a tax-6 (ok2065) mutant background (Log Rank, P=0.28 and P=0.73 respectively). b. tax-6 RNAi induced lifespan extension seen in wild type worms is completely suppressed in transgenic worms expressing constitutively nuclear CRTC-1 (S76A, S179A)::RFP. Lifespan of CRTC-1 (S76A, S179A)::RFP worms fed empty vector is not significantly different from wild type (Log Rank, P=0.07). Lifespan of CRTC-1 (S76A, S179A)::RF′P is not significantly different on empty vector or tax-6 RNAi (Log Rank, P=0.74). c. Suppression of tax-6 RNAi lifespan is specific to nuclear localised CRTC-1::RFP, since RNAi of tax-6 extends lifespan of worms expressing wild type CRTC-1:::RFP (Log rank, P<0.0001), but has no effect on the S76A, S179A double mutant (Log Rank P=0.57). There is no significant difference between lifespans of CRTC-1::RFP and CRTC-1 (S76A, S179A) fed empty vector (Log Rank, P=0.48).

FIG. 6 depicts CRTC-1 and CRH-1. a. Promoter regions of crtc-1 and crh-1 driving RFP (top panel, gray) or GFP (bottom panel, gray and light gray shades) respectively show expression in overlapping tissues. b. Co-IP in 293T cells transfected with FLAG tagged CRTC-1 and HA tagged CRH-1 showing C. elegans CRTC-1 and CRH-1 bind in vivo. Control blocking with excess FLAG peptide shows enrichment of HA::CRH-1 is FLAG CRTC-1 specific. c. RNAi of crh-1 either from hatch or day one of adulthood extended lifespan in the RNAi sensitive rrf-3 (pk1426) mutants, (Log Rank, P=0.003 and P=0.0001 respectively). d. RNAi of gluconeogenic genes increases wild type lifespan: PEPCK W05G11.6 (P<0.0001), PEPCK R11A5.4 (P<0.0001), PEPCK H04M03.1 (P<0.0001), gpi-1 (P<0.001), pyc-1 (P=0.01). Log Rank in each case. None of the RNAi treatments extended longevity by indirectly imposing dietary restriction since they did not affect feeding rates of C. elegans as measured by pumping rates of the pharynx (FIG. 11). e. Model depicting AMPK & TAX-6's antagonistic regulation of CRTC-1 and its effects on longevity. Mammalian orthologues in parentheses.

FIG. 7 shows: a. Amino acid sequence of CRTC-1. Translated sequence not contained in wormbase or genebank in unhighlighted bold (i.e., LQSPNHMMTPMYG). Serines 76 and 179 are identified in bold and highlighted gray (see second and fourth rows from top). Putative CREB binding domain determined by sequence homology to human CRTC2 underlined⁹. Putative NES sequences are bolded and highlighted very light gray (see penultimate and last rows): aa 420-427 and aa 446-454. Putative calcineurin binding site partially overlaps with NES1 and is highlighted in dark gray (see penultimate row). Putative 14-3-3 binding sites are highlighted in light gray (see second and fourth rows from top). b. Alignment of C. elegans CRTC-1 and human CRTC2 showing conserved phosphorylation sites at serines 76 & 179 with residues selected for in AMPK substrates shown in bold¹⁸. c. aak-2c promoter driving RFP shows aak-2c is expressed in intestine and head neurons. d. RNAi knockdown via feeding bacteria expressing crtc-1 double stranded RNA reduced crtc-1: :RFP expression in the intestine but does not deplete expression in the neurons.

FIG. 8 shows: aak-1 (tm1944); aak-2 (ok524) double mutants (aak −/−) are sensitive to heat stress but this sensitivity can be rescued by expression of truncated gain-of-function aak-2c (aa1-321), which confers heat resistance when expressed in wild type worms.

FIG. 9 shows: Heat stress results in nuclear exclusion of CRTC-1::RFP (top) but this effect in not seen in worms subjected to par-5 &fit-2 (14-3-3) RNAi, in which CRTC-1::RFP is constitutively nuclear. Arrows indicate nucleus.

FIG. 10 shows: Food intake, measured as rate of pharyngeal pumping, is not significantly affected by any of the RNAi treatments used (Wilcoxon Test Rank Sum, P=0.17).

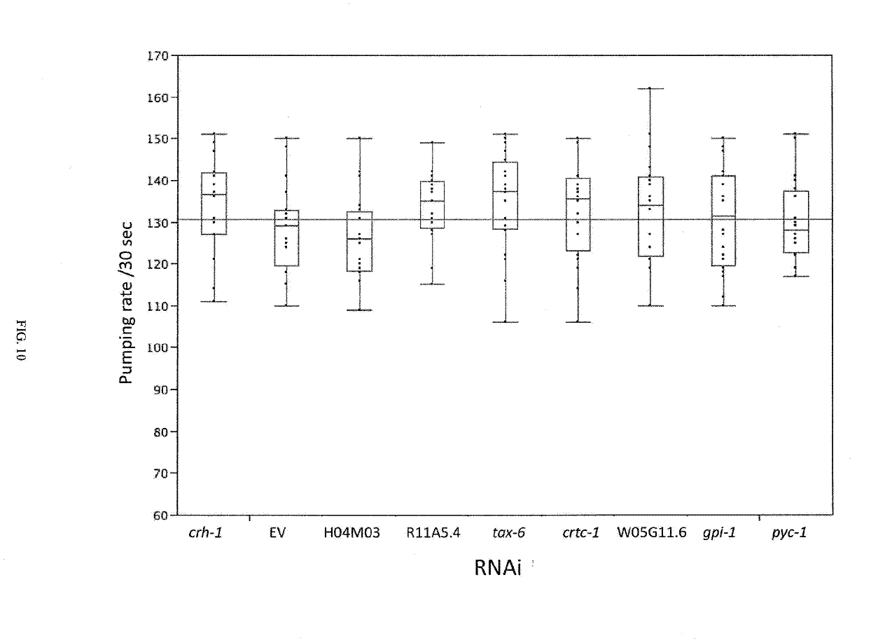

FIG. 11 shows: a. Wild type lifespan is increased by tax-6 RNAi either from hatch or from larval stage 4 (Log Rank P<0.0001 in both cases). b. rrf-3 (pk1426) lifespan is increased by tax-6 RNAi either from hatch or from larval stage 4 (Log Rank P<0.0001 in both cases). c. tax-6 RNAi has no significant effect on the lifespan of worms expressing CRTC-1::RFP(S76A, S179A), either from hatch (Log Rank, P=0.38) or from L4 (Log Rank, P=0.26).

FIG. 12 is a graph depicting additional CRTC-dependent transcription factors and their effect on longevity in the worm.

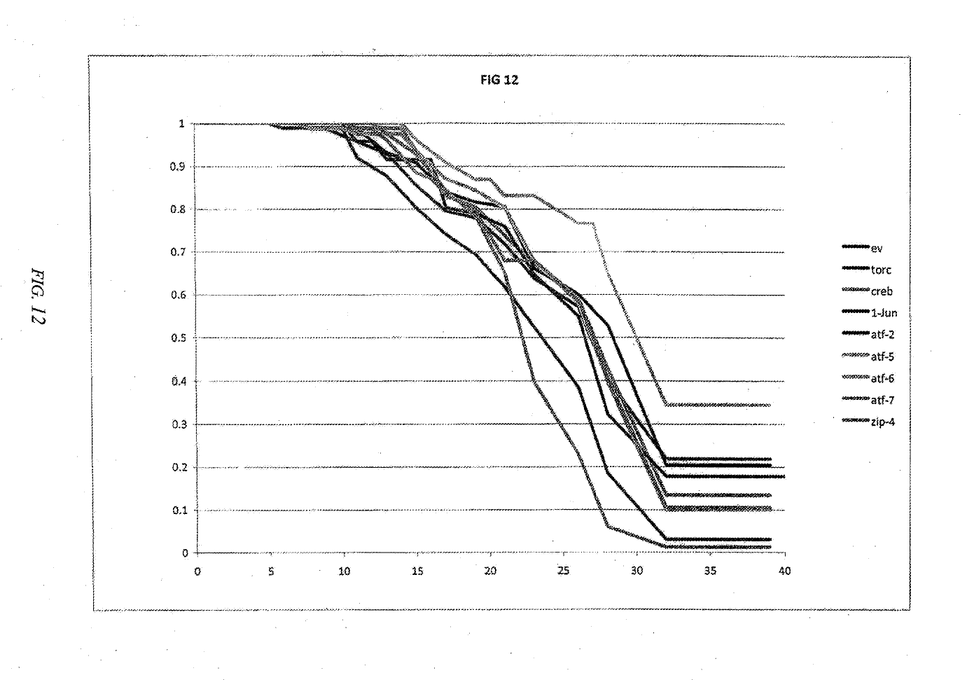

FIG. 13 depicts nucleic acid sequence encoding a CRTC-1 protein.

### DETAILED DESCRIPTION

In mammals, CRTCs (CREB regulated transcriptional coactivators) are a family of cofactors involved in energy homeostasis.

Applicants have identified the sole C. elegans CRTC family member, CRTC-1, and find that it is repressed by AMP-kinase activation, or de-activation of the phosphatase calcineurin, two conditions that extend longevity in the worm. Notably, Applicants have found that lifespan extension resulting from reduced calcineurin activity functions exclusively through CRTC-1 via conserved phosphorylation sites. In addition, reduction of CRTC-1 itself, its binding partner CREB homologue-1 (CRH-1) as well as CRH-1 target genes also slows aging. Taken together, these data demonstrate how the phosphorylation status of a single transcription cofactor can have profound effects upon energy homeostasis and ultimately organismal aging.

cAMP response element binding protein (CREB)-regulated transcriptional coactivators (CRTCs) represent a family of cofactors whose activity state is determined by energy status⁵⁷. Mammals have three CRTC family members, first identified as coactivators of CREB due to their ability to induce CREB target gene expression in the absence of a cAMP stimulus^{58,59}, and more recently reported to also interact with other transcription factors such as AP-1 and ATF-6^{40,12}. CRTCs bind as tetramers to the bZIP domain of CREB and facilitate recruitment of the transcriptional apparatus⁵⁸. CRTCs are negatively regulated through phosphorylation by AMP-activated protein kinase (AMPK) family kinases, including salt-inducible kinase 2 (SIK2)⁸, AMPK¹⁰ and microtubule affinity regulating kinase 2 (MARK2)²². Phosphorylation by these kinases facilitates 14-3-3 protein binding and retention of CRTCs in the cytoplasm⁸. Conversely, CRTCs are activated via dephosphorylation by the serine/threonine phosphatase calcineurin, which induces CRTC nuclear translocation and consequent activation of CREB targets^{8,20}. Recently, CRTC2 has been suggested to be a key target of AMPK in maintaining energy homeostasis via nutrient sensing in the hypothalamus³⁸.

In C. elegans, manipulations to both AMPK and calcineurin delay aging and are responsive to changes in nutritional status. AMPK is activated under low energy conditions when intracellular AMP:ATP ratios are high¹⁸. When active, AMPK functions to shut down key energy requiring processes such as growth, translation and gluconeogenesis via a range of targets including mTOR, SIRT1, CRTCs and FOXOs¹⁸ and functions as a nutrient sensor in the hypothalamus⁵⁹. C. elegans expressing gain of function (GOF) components of the AMPK heterotrimer are long-lived^{6,13}, and AMPK is required for the longevity of dauer larvae, a long-lived spore-like alternative larval stage induced in worms under low nutrient conditions³⁰. TAX-6 is the C. elegans orthologue of the calcineurin catalytic A subunit²⁶ and plays a key role in chemo/thermotaxis and growth²⁶, and is a target of IIS signalling⁷. In contrast to AMPK, tax-6 negatively regulates lifespan and loss of function (LOF) of tax-6 either by RNAi or genetic mutation results in long-lived worms⁷.

Applicants tested if the mechanism by which these separate longevity factors extend lifespan in response to changes in nutrition is by control of transcription through regulation of CRTC activity. If doing so, Applicants reasoned they might be able to uncover the conserved transcriptional targets that link the metabolic function of CRTCs and CREB to longevity assurance and demonstrate for the first time how nutritionally induced post translational changes to a cofactor can regulate aging.

As detailed herein, the results of the experiments presented herein illustrate how post-translational modification of a transcription cofactor can modulate transcriptional regulation of aging initiated by changes to nutritional status. Furthermore, Applicants have found that CRTC-1 activity can modulate aging and that CRTC-1 is a key longevity target of AMPK and calcineurin. Cofactor binding to transcription factors can both increase and repress expression of target genes, and this regulatory mechanism may also be involved in the pro-longevity effects of other transcription factors such as DAF-16, PHA-4 and SKN-1.

CRTC-1 represents a central node integrating different longevity pathways, since calcineurin is an IIS target and AMPK has been shown to be involved in diet restriction^{13,7}. Furthermore, gain of function of the histone deacetylase sir-2.1 increases lifespan in the worm⁶⁰, and in mice SIRT1 deactivates CRTC2 by deacetylation, allowing ubiquitination and degradation by the proteosome¹¹. Collectively, these data establish the conservation of this key pathway from worms to mammals.

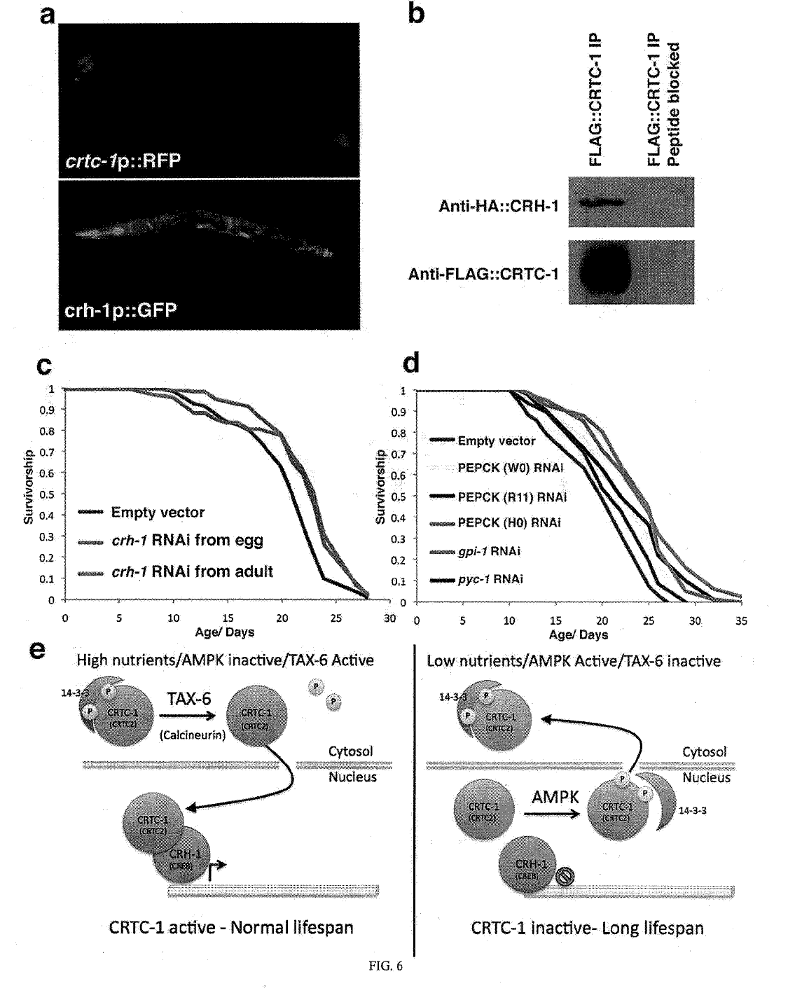

Results of the experiments presented herein identify a pivotal role for CREB regulated transcription coactivators in mediating longevity in response to environmental cues. In addition, Applicants find that pro-longevity effects of activating AMPK and inactivating TAX-6 converge to suppress CRTC-1 and that this is sufficient to slow ageing (FIG. 6e). Furthermore, Applicants identify CRTC-1 as the sole target of calcineurin mediated longevity and provide a novel role for CREB in controlling the balance between energy homeostasis and longevity assurance via regulation of its gluconeogenic target genes.

Collectively these data identify CRTC-1 as a central node by which an organism's nutritional and environmental status can mediate its rate of ageing, linking AMPK and calcineurin via a shared signalling transduction pathway and demonstrating that post-translational modification of a single transcriptional cofactor can significantly modulate longevity. As is true for both insulin and TOR signalhng^{3,4,5}, work in the worm can greatly advance knowledge of conserved longevity pathways and Applicants have established C. elegans as a complementary model system that is directly applicable to mammalian studies of CRTCs. Applicants' data, coupled with recent findings on the health benefits of reducing components of the CRTC pathway in mice^{10,35,36}, highlight CRTCs not only as novel ageing regulators but also as potential therapeutic targets for patients with metabolic disorders.

Mammals CRTCs are expressed in multiple tissues including brain and liver and both CRTC1 and 2 are involved in energy balance^{10,11,37}. Additionally, the same CRTC can have separable roles in distinct tissue types; CRTC2 regulates energy homeostasis in the liver¹⁰ and has been shown to be an energy sensor in the hypothalamus³⁸, the area of the brain responsible for nutrient sensing. The sole C. elegans CRTC may therefore fulfil multiple roles that have diverged during mammalian evolution. CRTC-1 is expressed in the nerve ring in the head of C. elegans and the intestine, which, along with digestive function also plays an endocrine ‘liver-like’ role in the worm. Identifying the spatial and temporal requirements for the longevity effects of CRTC-1 will therefore be vital to understanding its mode of action.

Although reducing crh-1 expression increased lifespan, this effect was not as great as those seen by reductions to the upstream components tax-6 or crtc-1. CRTC family members were first identified as coactivators of CREB due to their ability to induce CREB target gene expression in the absence of a cAMP stimulus^{9,39}, but recently have been reported to also interact with other transcription factors such as AP-1 and ATF-6^{12,40}. Without wishing to be bound to theory, Applicants believe CRTC-1 may therefore complex with other transcription factors besides CRH-1 in the worm, the targets of which may contribute to the lifespan effects. Alternatively, CRTC-1 may have cytosolic function enhanced by its confinement to the cytoplasm during tax-6 RNAi, but not seen in knockdown of either crtc-1 or crh-1. Such a bifunctional role has recently been shown for the mammalian diabetes and obesity regulated (DOR) protein, which acts in the nucleus as a cofactor for the hormone receptor TRα1, but shuttles to the cytosol under starvation where it enhances autophagy and protein degradation⁴¹. Similarly, p53, a key transcription factor implicated in tumorigenesis and ageing has recently been shown to have distinct cytoplasmic functions separable from its nuclear role⁴², including inhibition of autophagy via AMPK and mTOR⁴³.

Both diet restriction and reduced IIS have been reported to increase transcription/activity of gluconeogenic genes^{44,45,46,47}, making it counter-intuitive that reducing levels of gluconeogenic enzymes increases lifespan. However, starvation does not increase expression of the PEPCKs R11A5.4 or W05G11.6 in adult worms⁴⁸ and in mice, although fasting initially activates glucose production in the liver, gluconeogenesis is later diminished after prolonged nutrient deprivation via the inhibition of CRTC2 by AMPK and SIRT^{10,11}. RNAi for at least one PEPCK (R11A5.4) has previously been shown to further increase the lifespan of long-lived worms carrying mutations to the gene encoding the insulin/IGF-1-like receptor DAF-2⁷, while a whole genome RNAi screen for genes that increased C. elegans lifespan identified gpi-1⁴⁹. Moreover, feeding worms glucose shortens lifespan⁵⁰, while glucose restriction extends it⁵¹.

The data presented herein demonstrate a new level of complexity in the way an organism regulates its rate of ageing in response to the environment and have broader implications for the ability to manipulate only the pro-longevity functions of other transcription factors such as DAF-16, HSF-1, PHA-4 and SKN-1¹. For example, the effect of DAF-16 on reproduction, dauer formation and longevity can be uncoupled⁵², raising the possibility that regulation by alternative cofactors separates the roles of DAF-16 in each of these physiological processes.

An aim of ageing research is to distinguish between the pro-health effects of long-lived mutants and their detrimental pleiotropic side effects, in order to treat age-related disorders. In certain embodiments, targeting interventions that modify the activity of specific cofactors may therefore allow translating ageing research into treatments that yield only positive therapeutic effects.

As used herein, the term “amino acid sequence” is synonymous with the terms “polypeptide,” “protein,” and “peptide,” and are used interchangeably. Where such amino acid sequences exhibit activity, they may be referred to as an “enzyme.” The conventional one-letter or three-letter code for amino acid residues are used herein.

The term “nucleic acid” encompasses DNA, RNA, heteroduplexes, and synthetic molecules capable of encoding a polypeptide and includes all analogs and backbone substitutes such as PNA that one of ordinary skill in the art would recognize as capable of substituting for naturally occurring nucleotides and backbones thereof. Nucleic acids may be single stranded or double stranded, and may be chemical modifications. The terms “nucleic acid” and “polynucleotide” are used interchangeably. Because the genetic code is degenerate, more than one codon may be used to encode a particular amino acid, and the present compositions and methods encompass nucleotide sequences which encode a particular amino acid sequence.

Unless otherwise indicated, nucleic acids are written left to right in 5′ to 3′ orientation; amino acid sequences are written left to right in amino to carboxy orientation, respectively.

As used herein, “hybridization” refers to the process by which one strand of nucleic acid base pairs with a complementary strand, as occurs during blot hybridization techniques and PCR techniques.

Hybridization conditions are based on the melting temperature (Tm) of the nucleic acid binding complex, as taught, e.g., in Berger and Kimmel (1987, Guide to Molecular Cloning Techniques, Methods in Enzymology, Vol 152, Academic Press, San Diego Calif.), and confer a defined “stringency” as explained below.

Maximum stringency typically occurs at about Tm-5° C. (5° C. below the Tm of the probe); high stringency at about 5° C. to 10° C. below Tm; intermediate stringency at about 10° C. to 20° C. below Tm; and low stringency at about 20° C. to 25° C. below Tm. As will be understood by those of ordinary skill in the art, a maximum stringency hybridization can be used to identify or detect identical nucleotide sequences while an intermediate (or low) stringency hybridization can be used to identify or detect similar or related polynucleotide sequences.

In one aspect, the present invention covers nucleotide sequences that can hybridize to another nucleotide sequence under stringent conditions (e.g., 65° C. and 0.1×SSC {1×SSC=0.15 M NaCl, 0.015 M Na3 Citrate pH 7.0). Where the nucleotide sequence is double-stranded, both strands of the duplex, either individually or in combination, are encompassed by the present invention. Where the nucleotide sequence is single-stranded, it is to be understood that the complementary sequence of that nucleotide sequence is also included within the scope of the present invention.

Stringency of hybridization refers to conditions under which polynucleic acid hybrids are stable. Such conditions are evident to those of ordinary skill in the field. As known to those of ordinary skill in the art, the stability of hybrids is reflected in the melting temperature (Tm) of the hybrid which decreases approximately 1 to 1.5° C. with every 1% decrease in sequence homology. In general, the stability of a hybrid is a function of sodium ion concentration and temperature. Typically, the hybridization reaction is performed under conditions of higher stringency, followed by washes of varying stringency.

As used herein, high stringency includes conditions that permit hybridization of only those nucleic acid sequences that form stable hybrids in 1 M Na+ at 65-68° C. High stringency conditions can be provided, for example, by hybridization in an aqueous solution containing 6×SSC, 5×Denhardt's, 1% SDS (sodium dodecyl sulphate), 0.1 Na⁺ pyrophosphate and 0.1 mg/ml denatured salmon sperm DNA as non specific competitor. Following hybridization, high stringency washing may be done in several steps, with a final wash (about 30 min) at the hybridization temperature in 0.2-0.1×SSC, 0.1% SDS.

It is understood that these conditions may be adapted and duplicated using a variety of buffers, e.g., formamide-based buffers, and temperatures. Denhardt's solution and SSC are well known to those of ordinary skill in the art as are other suitable hybridization buffers (see, e.g., Sambrook, et al., eds. (1989) Molecular Cloning: A Laboratory Manual, Cold Spring Harbor Laboratory Press, New York or Ausubel, et al., eds. (1990) Current Protocols in Molecular Biology, John Wiley & Sons, Inc.). Optimal hybridization conditions have to be determined empirically, as the length and the GC content of the hybridizing pair also play a role.

As used herein, a “synthetic” molecule is produced by in vitro chemical or enzymatic synthesis rather than by an organism.

The term “heterologous” with reference to a polynucleotide or protein refers to a polynucleotide or protein that does not naturally occur in a host cell.

As used herein, the term “expression” refers to the process by which a polypeptide is produced based on the nucleic acid sequence of a gene. The process includes both transcription and translation.

A “gene” refers to the DNA segment encoding a polypeptide.

“Antisense” nucleic acids are DNA or RNA molecules that are complementary to at least a portion of a specific mRNA molecule (Weintraub, Scientific American 262 40, 1990). In the cell, the antisense nucleic acids hybridize to the corresponding mRNA, forming a double-stranded molecule. This interferes with the translation of the mRNA since the cell will not translate an mRNA that is double-stranded. Antisense oligomers of at least about 15, about 20, about 25, about 30, about 35, about 40, or of at least about 50 nucleotides are preferred, since they are easily synthesized and are less likely to cause non-specific interference with translation than larger molecules. The use of antisense methods to inhibit the in vitro translation of genes is well known in the art (Marcus-Sakura Anal. Biochem. 172: 289, 1998). In the present case, animals transformed with constructs containing antisense fragments of a CRTC gene, such as for example, CRTC-1 in Caenorhabditis elegans and homologs thereof, would display a modulated phenotype such as altered longevity.

The invention provides for nucleic acids complementary to (e.g., antisense sequences to) cellular modulators of CRTC activity. Antisense sequences are capable of inhibiting the transport, splicing or transcription of protein-encoding genes, e.g., nucleic acids encoding CRTC-1 in Caenorhabditis elegans. The inhibition can be effected through the targeting of genomic DNA or messenger RNA. The transcription or function of targeted nucleic acid can be inhibited, for example, by hybridization and/or cleavage. One particularly useful set of inhibitors provided by the present invention includes oligonucleotides which are able to either bind gene or message, in either case preventing or inhibiting the production or function of the protein. The association can be through sequence specific hybridization. Another useful class of inhibitors includes oligonucleotides that cause inactivation or cleavage of protein message. The oligonucleotide can have enzyme activity which causes such cleavage, such as ribozymes. The oligonucleotide can be chemically modified or conjugated to an enzyme or composition capable of cleaving the complementary nucleic acid. One can screen a pool of many different such oligonucleotides for those with the desired activity.

Short double-stranded RNAs (dsRNAs; typically <30 nucleotides) can be used to silence the expression of target genes in animals and animal cells. Upon introduction, the long dsRNAs enter the RNA interference (RNAi) pathway which involves the production of shorter (20-25 nucleotide) small interfering RNAs (siRNAs) and assembly of the siRNAs into RNA-induced silencing complexes (RISCs). The siRNA strands are then unwound to form activated RISCs, which cleave the target RNA. Double stranded RNA has been shown to be extremely effective in silencing a target RNA. Introduction of double stranded RNA corresponding to, e.g., a CRTC gene, would be expected to modify the CRTC-related functions discussed herein including, but not limited to, longevity.

General methods of using antisense, ribozyme technology and RNAi technology, to control gene expression, or of gene therapy methods for expression of an exogenous gene in this manner are well known in the art. Each of these methods utilizes a system, such as a vector, encoding either an antisense or ribozyme transcript. The term “RNAi” stands for RNA interference. This term is understood in the art to encompass technology using RNA molecules that can silence genes. See, for example, McManus, et al. Nature Reviews Genetics 3: 737, 2002. In this application, the term “RNAi” encompasses molecules such as short interfering RNA (siRNA), small hairpin or short hairpin RNA (smRNA), microRNAs, and small temporal RNA (stRNA). Generally speaking, RNA interference results from the interaction of double-stranded RNA with genes.

“Small interfering RNA” (siRNA) refers to double-stranded RNA molecules from about 10 to about 30 nucleotides long that are named for their ability to specifically interfere with protein expression through RNA interference (RNAi). Preferably, siRNA molecules are 12-28 nucleotides long, more preferably 15-25 nucleotides long, still more preferably 19-23 nucleotides long and most preferably 21-23 nucleotides long. Therefore, preferred siRNA molecules are 12, 13, 14, 15, 16, 17, 18, 19, 20, 21, 22, 23, 24, 25, 26, 27 28 or 29 nucleotides in length.

RNAi is a two-step mechanism (Elbashir et al., Genes Dev., 15: 188-200, 2001). First, long dsRNAs are cleaved by an enzyme known as Dicer in 21-23 ribonucleotide (nt) fragments, called small interfering RNAs (siRNAs). Then, siRNAs associate with a ribonuclease complex (termed RISC for RNA Induced Silencing Complex) which target this complex to complementary mRNAs. RISC then cleaves the targeted mRNAs opposite the complementary siRNA, which makes the mRNA susceptible to other RNA degradation pathways.

siRNAs of the present invention are designed to interact with a target ribonucleotide sequence, meaning they complement a target sequence sufficiently to bind to the target sequence. The present invention also includes siRNA molecules that have been chemically modified to confer increased stability against nuclease degradation, but retain the ability to bind to target nucleic acids that may be present.

The invention provides antisense oligonucleotides capable of binding messenger RNA, e.g., mRNA encoding CRTC-1 in C. elegans, that can inhibit polypeptide activity by targeting mRNA. Strategies for designing antisense oligonucleotides are well described in the scientific and patent literature, and the ordinarily skilled artisan can design such oligonucleotides using the novel reagents of the invention. For example, gene walking/RNA mapping protocols to screen for effective antisense oligonucleotides are well known in the art, see, e.g., Ho, Methods Enzymol. 314: 168-183, 2000, describing an RNA mapping assay, which is based on standard molecular techniques to provide an easy and reliable method for potent antisense sequence selection. See also Smith, Eur. J. Pharm. Sci. 11: 191-198, 2000.

Naturally occurring nucleic acids are typically used as antisense oligonucleotides. The antisense oligonucleotides can be of any length; for example, in alternative aspects, the antisense oligonucleotides are between about 5 to 100, about 10 to 80, about 15 to 60, about 18 to 40. The optimal length can be determined by routine screening. The antisense oligonucleotides can be present at any concentration. The optimal concentration can be determined by routine screening. A wide variety of synthetic, non-naturally occurring nucleotide and nucleic acid analogues are known which can also be used. For example, peptide nucleic acids (PNAs) containing non-ionic backbones, such as N-(2-aminoethyl) glycine units can be used. Antisense oligonucleotides having phosphorothioate linkages can also be used, as described in Mata, Toxicol Appl Pharmacol. 144: 189-197, 1997; Antisense Therapeutics, ed. Agrawal, Humana Press, Totowa, N.J., 1996. Antisense oligonucleotides having synthetic DNA backbone analogues can also include phosphoro-dithioate, methylphosphonate, phosphoramidate, alkyl phosphotriester, sulfamate, 3′-thioacetal, methylene(methylimino), 3′-N-carbamate, and morpholino carbamate nucleic acids, as described above.

Combinatorial chemistry methodology can be used to create vast numbers of oligonucleotides that can be rapidly screened for specific oligonucleotides that have appropriate binding affinities and specificities toward any target, such as the sense and antisense polypeptides sequences of the invention (see, e.g., Gold, J. of Biol. Chem. 270: 13581-13584, 1995).

By “homolog” is meant an entity having a certain degree of identity with the subject amino acid sequences and the subject nucleotide sequences. As used herein, the term “homolog” covers identity with respect to structure and/or function, for example, the expression product of the resultant nucleotide sequence has the inhibitory or upregulatory activity of a subject amino acid sequence. With respect to sequence identity, preferably there is at least 70%, 75%, 80%, 81%, 82%, 83%, 84%, 85%, 86%, 87%, 88%, 89%, 90%, 91%, 92%, 93%, 94%, 95%, 96%, 97%, 98%, or even 99% sequence identity. These terms also encompass allelic variations of the sequences. The term, homolog, may apply to the relationship between genes separated by the event of speciation or to the relationship between genes separated by the event of genetic duplication.

Relative sequence identity can be determined by commercially available computer programs that can calculate % identity between two or more sequences using any suitable algorithm for determining identity, using for example default parameters. A typical example of such a computer program is CLUSTAL. Advantageously, the BLAST algorithm is employed, with parameters set to default values. The BLAST algorithm is described in detail at http://www.ncbi.nih.gov/BLAST/blast_help.html, which is incorporated herein by reference.

The homologs of the peptides as provided herein typically have structural similarity with such peptides. A homolog of a polypeptide includes one or more conservative amino acid substitutions, which may be selected from the same or different members of the class to which the amino acid belongs.

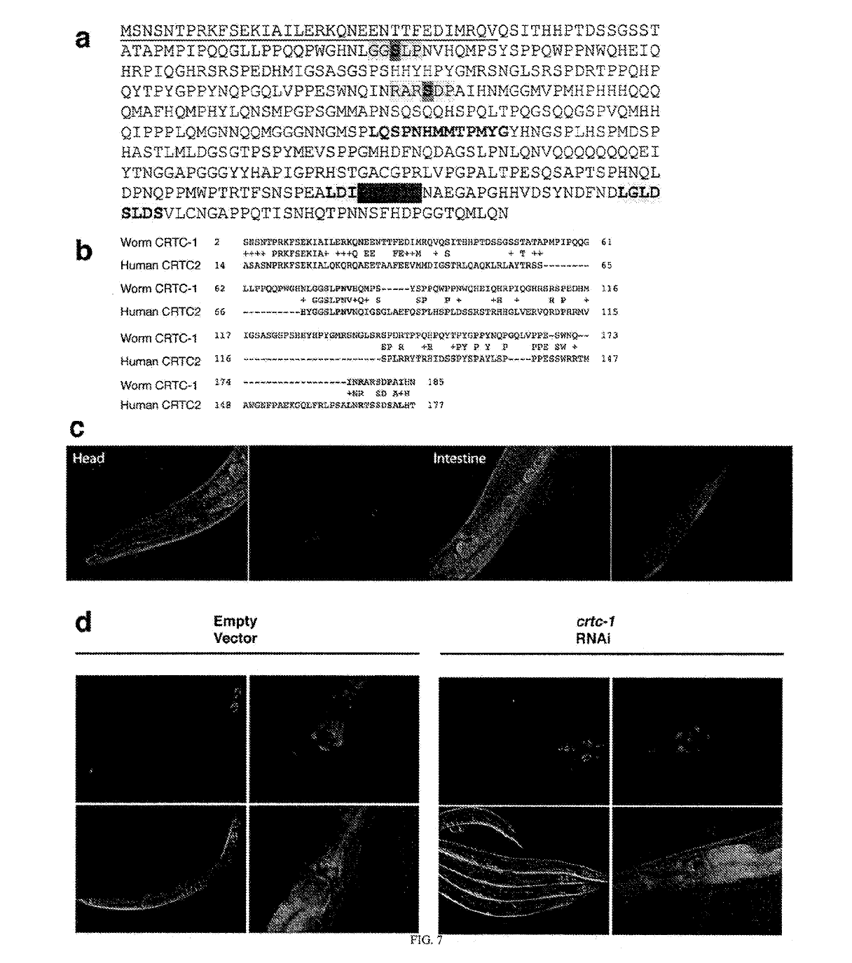

Thus, in certain embodiments, the present invention also encompasses the use of variants, homologues and derivatives of the CRTC-1 amino acid sequence in FIG. 7a.

In one embodiment, the sequences, such as variants, homologs and derivatives of the CRTC-1 amino acid sequence in FIG. 7a, may also have deletions, insertions or substitutions of amino acid residues which produce a silent change and result in a functionally equivalent substance. Deliberate amino acid substitutions may be made on the basis of similarity in polarity, charge, solubility, hydrophobicity, hydrophilicity, and/or the amphipathic nature of the residues as long as the secondary binding activity of the substance is retained. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

The present invention also encompasses conservative substitution (substitution and replacement are both used herein to mean the interchange of an existing amino acid residue with an alternative residue) that may occur e.g., like-for-like substitution such as basic for basic, acidic for acidic, polar for polar, etc. Non-conservative substitution may also occur e.g., from one class of residue to another or alternatively involving the inclusion of unnatural amino acids such as ornithine (hereinafter referred to as Z), diaminobutyric acid ornithine (hereinafter referred to as B), norleucine ornithine (hereinafter referred to as O), pyriylalanine, thienylalanine, naphthylalanine and phenylglycine. Conservative substitutions that may be made are, for example within the groups of basic amino acids (Arginine, Lysine and Histidine), acidic amino acids (glutamic acid and aspartic acid), aliphatic amino acids (Alanine, Valine, Leucine, Isoleucine), polar amino acids (Glutamine, Asparagine, Serine, Threonine), aromatic amino acids (Phenylalanine, Tryptophan and Tyrosine), hydroxylamino acids (Serine, Threonine), large amino acids (Phenylalanine and Tryptophan) and small amino acids (Glycine, Alanine).

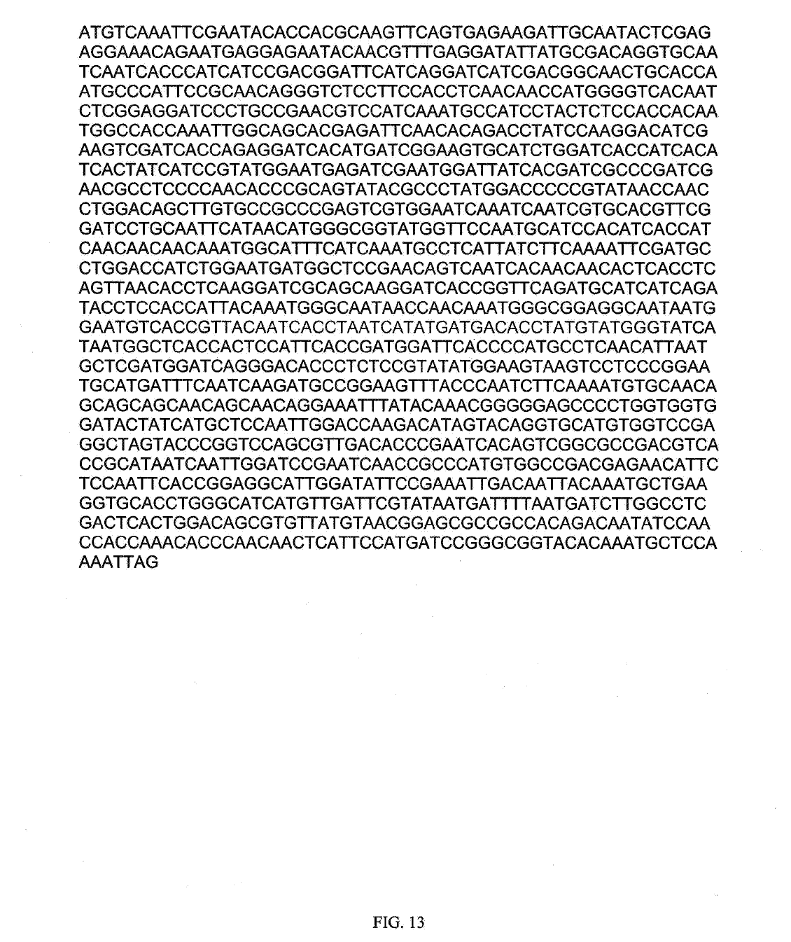

Examples of homologs according to the invention include CRTC-1 homologs, such as nucleotides with at least 70%, at least 80%, at least 90%, at least 95%, at least 98% sequence identity to the nucleotide sequence depicted in FIG. 13.

Examples of homologs according to the invention also include aak-2 (ampk alpha subunit) homologs, such as nucleotides with at least 70%, at least 80%, at least 90%, at least 95%, at least 98% sequence identity to the nucleotide sequence depicted in GenBank Accession No. NM_{—}078309. Examples of homologs according to the invention also include tax-6 (calcineurin catalytic subunit) homologs, such as nucleotides with at least 70%, at least 80%, at least 90%, at least 95%, at least 98% sequence identity to the nucleotide sequence depicted in GenBank Accession No. NM_{—}001083189. Examples of homologs according to the invention also include crh-1 homologs, such as nucleotides with at least 70%, at least 80%, at least 90%, at least 95%, at least 98% sequence identity to the nucleotide sequence depicted in GenBank Accession No. NM_{—}001027688.

Examples of homologs according to the invention also include ubl-5 homologs, such as peptides with at least 70%, more preferably at least 80%, more preferably at least 90%, more preferably at least 95%, more preferably at least 98% sequence identity to the amino acid sequence depicted in GenBank Accession No. NP_{—}491640.

Examples of homologs according to the invention also include aak-2 (ampk alpha subunit) homologs, such as peptides with at least 70%, at least 80%, at least 90%, at least 95%, at least 98% sequence identity to the nucleotide sequence depicted in GenBank Accession No. NP_{—}510710. Examples of homologs according to the invention also include tax-6 (calcineurin catalytic subunit) homologs, such as peptides with at least 70%, at least 80%, at least 90%, at least 95%, at least 98% sequence identity to the nucleotide sequence depicted in GenBank Accession No. NP_{—}001076658. Examples of homologs according to the invention also include crh-1 homologs, such as peptides with at least 70%, at least 80%, at least 90%, at least 95%, at least 98% sequence identity to the nucleotide sequence depicted in GenBank Accession No. NP_{—}001022859.

In yet other embodiments, homologs according to the invention include CRTC-1 homologs, such as peptides with at least 70%, at least 80%, at least 90%, at least 95%, at least 98% sequence identity to the amino acid sequence depicted in FIG. 7a.

Another embodiment of the invention relates to animals that have at least one modulated CRTC function. Such modulated functions include among others an altered longevity. “Longevity” refers to the life span of an animal. Thus, longevity refers to the number of years in the life span of an animal. “Stress tolerance” refers to an animal's ability to tolerate exposure to various internal and external environmental challenges such as exposure to UV light, exposure to high osmolarity, exposure to infection, exposure to oxidative damage, exposure to metal compounds, and exposure to certain toxins. Those of ordinary skill in the art will recognize that an increase in the lifespan of an animal can readily be measured by various assays known in the art. The field of gerontology is one such example of a relevant art. By way of example, longevity may be assessed by various markers such as number of generations to senescence in non-immortalized somatic cells, graying hair, wrinkling, and other such alterations in physiological markers associated with aging. Those of ordinary skill in the art will also recognize that alterations in an animal's ability to tolerate stress, i.e., its response to stress, may be assessed by various assays, including by way of example, by assessing changes in expression or activity of molecules involved in the stress response by measuring expression of stress response genes, protein levels of specific stress response proteins, or activity levels of specific stress response proteins.

Animals having a modified CRTC-related function include transgenic animals with an altered longevity due to transformation with constructs using antisense or siRNA technology that affect transcription or expression from a CRTC, AMPK, and/or calcineurin gene. Such animals exhibit an altered longevity.

Accordingly, in another series of embodiments, the present invention provides methods of screening or identifying proteins, small molecules or other compounds which are capable of inducing or inhibiting the activity or expression of CRTC, AMPK, and/or calcineurin genes and proteins. The assays may be performed, by way of example, in vitro using transformed or non-transformed cells, immortalized cell lines, or in vivo using transformed animal models enabled herein.

To aid in the detection of a protein or nucleic acid, labels are typically used—such as any readily detectable reporter, for example, a fluorescent, bioluminescent, phosphorescent, radioactive, etc. reporter. For example, labels suitable for use in the methods and compositions of the instant invention include green fluorescent protein, yellow fluorescent protein, blue fluorescent protein, and red fluorescent protein. Examples of such reporters (e.g., green fluorescent protein, red fluorescent protein), their detection, coupling to targets/probes, etc. are disclosed herein, for example, in the non-limiting examples.

The present invention further contemplates direct and indirect labelling techniques. For example, direct labelling includes incorporating fluorescent dyes directly into a nucleotide sequence (e.g., dyes are incorporated into nucleotide sequence by enzymatic synthesis in the presence of labelled nucleotides or PCR primers). Direct labelling schemes include using families of fluorescent dyes with similar chemical structures and characteristics. In certain embodiments comprising direct labelling of nucleic acids, cyanine or alexa analogs are utilized. In other embodiments, indirect labelling schemes can be utilized, for example, involving one or more staining procedures and reagents that are used to label a protein in a protein complex (e.g., a fluorescent molecule that binds to an epitope on a protein in the complex, thereby providing a fluorescent signal by virtue of the conjugation of dye molecule to the epitope of the protein).

In another series of embodiments, the present invention provides methods for identifying proteins and other compounds which bind to, or otherwise directly interact with a CRTC protein. Thus, in one series of embodiments, High Throughput Screening-derived proteins, DNA chip arrays, cell lysates or tissue homogenates may be screened for proteins or other compounds which bind to one of the normal or mutant CRTC genes. Alternatively, any of a variety of exogenous compounds, both naturally occurring and/or synthetic (e.g., libraries of small molecules or peptides), may be screened for CRTC function modulating capacity.

Embodiments of the invention also include methods of identifying proteins, small molecules and other compounds capable of modulating the activity of a CRTC gene or protein. Using normal cells or animals, the transformed cells and animal models of the present invention, or cells obtained from subjects bearing normal or mutant CRTC genes, the present invention provides methods of identifying such compounds on the basis of their ability to affect the expression of a CRTC, the activity of a CRTC, the activity of proteins that interact with normal or mutant CRTC proteins, or other biochemical, histological, or physiological markers that distinguish cells bearing normal and modulated CRTC activity in animals.

In accordance with another aspect of the invention, the proteins of the invention can be used as starting points for rational chemical design to provide ligands or other types of small chemical molecules. Alternatively, small molecules or other compounds identified by the above-described screening assays may serve as “lead compounds” in design of modulators of CRTC-related traits, such as longevity, in animals.

DNA sequences encoding a CRTC protein can be expressed in vitro by DNA transfer into a suitable host cell. “Host cells” are cells in which a vector can be propagated and its DNA expressed. The term also includes any progeny or graft material, for example, of the subject host cell. It is understood that all progeny may not be identical to the parental cell since there may be mutations that occur during replication. However, such progeny are included when the term “host cell” is used. Methods of stable transfer, meaning that the foreign DNA is continuously maintained in the host, are known in the art.

The terms “recombinant expression vector” or “expression vector” refer to a plasmid, virus or other vehicle known in the art that has been manipulated by insertion or incorporation of a genetic sequence. Such expression vectors contain a promoter sequence which facilitates the efficient transcription of the inserted sequence. The expression vector typically contains an origin of replication, a promoter, as well as specific genes that allow phenotypic selection of the transformed cells.

Methods that are well known to those ordinarily skilled in the art can be used to construct expression vectors containing a CRTC coding sequence and appropriate transcriptional/translational control signals. These methods include in vitro recombinant DNA techniques, synthetic techniques, and in vivo recombination/genetic techniques.

A variety of host-expression vector systems may be utilized to express a coding sequence. These include but are not limited to microorganisms such as bacteria transformed with recombinant bacteriophage DNA, plasmid DNA or cosmid DNA expression vectors containing a coding sequence; yeast transformed with recombinant yeast expression vectors containing a coding sequence; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing a coding sequence; insect cell systems infected with recombinant virus expression vectors (e.g., baculovirus) containing a coding sequence; or animal cell systems infected with recombinant virus expression vectors (e.g., retroviruses, adenovirus, vaccinia virus) containing a coding sequence, or transformed animal cell systems engineered for stable expression.

Depending on the host/vector system utilized, any of a number of suitable transcription and translation elements, including constitutive and inducible promoters, transcription enhancer elements, transcription terminators, etc. may be used in the expression vector (see e.g., Bitter et al. Methods in Enzymology 153, 516-544, 1987). For example, when cloning in bacterial systems, inducible promoters such as pL of bacteriophage 7, plac, ptrp, ptac-(ptrp-lac hybrid promoter) and the like may be used. When cloning in mammalian cell systems, promoters derived from the genome of mammalian cells (e.g., metallothionein promoter) or from mammalian viruses (e.g., the retrovirus long terminal repeat; the adenovirus late promoter; the vaccinia virus 7.5K promoter) may be used.

The term “operably linked” refers to functional linkage between a promoter sequence and a nucleic acid sequence regulated by the promoter. The operably linked promoter controls the expression of the nucleic acid sequence.

The expression of structural genes may be driven by a number of promoters. Although the endogenous, or native promoter of a structural gene of interest may be utilized for transcriptional regulation of the gene, preferably, the promoter is a foreign regulatory sequence. For mammalian expression vectors, promoters capable of directing expression of the nucleic acid preferentially in a particular cell type may be used (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Banerji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546).

Promoters useful in the invention include both natural constitutive and inducible promoters as well as engineered promoters. Examples of inducible promoters useful in animals include those induced by chemical means, such as the yeast metallothionein promoter, which is activated by copper ions (Mett, et al. Proc. Natl. Acad. Sci., U.S.A. 90, 4567, 1993); and the GRE regulatory sequences which are induced by glucocorticoids (Schena, et al. Proc. Natl. Acad. Sci., U.S.A. 88, 10421, 1991). Other promoters, both constitutive and inducible will be known to those of ordinary skill in the art.

Animals included in the invention are any animals amenable to transformation techniques, including vertebrate and non-vertebrate animals and mammals. Examples of mammals include, but are not limited to, pigs, cows, sheep, horses, cats, dogs, chickens, or turkeys.

Compounds tested as modulators of CRTC activity can be any small organic molecule, or a biological entity, such as a protein, e.g., an antibody or peptide, a sugar, a nucleic acid, e.g., an antisense oligonucleotide, RNAi, or a ribozyme, or a lipid. Alternatively, modulators can be genetically altered versions of a cellular modulator of CRTC activity. Typically, test compounds will be small organic molecules, nucleic acids, peptides, lipids, and lipid analogs.

Essentially any chemical compound can be used as a potential modulator or ligand in the assays of the invention, although most often compounds can be dissolved in aqueous or organic solutions. In certain embodiments, the assays of the invention are designed to screen large chemical libraries by automating the assay steps and providing compounds from any convenient source to assays, which are typically run in parallel (e.g., in microtiter formats on microtiter plates in robotic assays). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs Switzerland) and the like.

In one embodiment, high throughput screening methods involve providing a combinatorial small organic molecule or peptide library containing a large number of potential therapeutic compounds (potential modulator or ligand compounds). Such “combinatorial chemical libraries” or “ligand libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37: 487-493, 1991 and Houghton et al., Nature 354: 84-88, 1991). Other chemistries for generating chemical diversity libraries can also be used. Such chemistries include, but are not limited to: peptoids (e.g., PCT Publication No. WO 91/19735), encoded peptides (e.g., PCT Publication No. WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90: 6909-6913, 1993), vinylogous polypeptides (Hagihara et al., J. Amer. Chem. Soc. 114: 6568, 1992), nonpeptidal peptidomimetics with glucose scaffolding (Hirschmann et al., J. Amer. Chem. Soc. 114: 9217-9218, 1992), analogous organic syntheses of small compound libraries (Chen et al., J. Amer. Chem. Soc. 116: 2661, 1994), oligocarbamates (Cho et al., Science 261: 1303, 1993), and/or peptidyl phosphonates (Campbell et al., J. Org. Chem. 59: 658, 1994), nucleic acid libraries (see Ausubel, Berger and Sambrook, all supra), peptide nucleic acid libraries (see, e.g., U.S. Pat. No. 5,539,083), antibody libraries (see, e.g., Vaughn et al, Nature Biotechnology, 14: 309-314, 1996 and PCT/US96/10287), carbohydrate libraries (see, e.g., Liang et al., Science 274: 1520-1522, 1996 and U.S. Pat. No. 5,593,853), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like.

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, R U, 3D Pharmaceuticals, Exton, Pa., Martek Biosciences, Columbia, Md., etc.).

Candidate compounds are useful as part of a strategy to identify drugs for enhancing longevity wherein the compounds modulate activity of cellular molecules regulated by the CRTC, for example, wherein the compound modulates the activity of CRH-1 or a homolog thereof. Screening assays for identifying candidate or test compounds that bind to one or more cellular modulators of CRTC activity, or polypeptides or biologically active portions thereof, are also included in the invention. The test compounds can be obtained using any of the numerous approaches in combinatorial library methods known in the art, including, but not limited to, biological libraries; spatially addressable parallel solid phase or solution phase libraries; synthetic library methods requiring deconvolution; the “one-bead one-compound” library method; and synthetic library methods using affinity chromatography selection. The biological library approach can be used for, e.g., peptide libraries, while the other four approaches are applicable to peptide, non-peptide oligomer or small molecule libraries of compounds (Lam, Anticancer Drug Des. 12: 145, 1997). Examples of methods for the synthesis of molecular libraries can be found in the art, for example in: DeWitt et al., Proc. Natl. Acad. Sci. U.S.A. 90: 6909, 1993; Erb et al., Proc. Natl. Acad. Sci. USA 91: 11422, 1994; Zuckermann et al., J. Med. Chem. 37: 2678, 1994; Cho et al., Science 261: 1303, 1993; Carrell et al., Angew. Chem. Int. Ed. Engl. 33: 2059, 1994; Caren et al., Angew. Chem. Int. Ed. Engl. 33: 2061, 1994; and Gallop et al., J. Med. Chem. 37: 1233, 1994.

Libraries of compounds can be presented in solution (e.g., Houghten, Bio/Techniques 13: 412-421, 1992), or on beads (Lam, Nature 354: 82-84, 1991), chips (Fodor, Nature 364: 555-556, 1993), bacteria (U.S. Pat. No. 5,223,409), spores (U.S. Pat. Nos. 5,571,698, 5,403,484, and 5,223,409), plasmids (Cull et al., Proc. Natl. Acad. Sci. USA 89: 1865-1869, 1992) or on phage (Scott et al., Science 249: 386-390, 1990; Devlin, Science 249: 404-406, 1990; Cwirla et al., Proc. Natl. Acad. Sci. USA 87: 6378-6382, 1990; and Felici, J. Mol. Biol. 222: 301-310, 1991).

This invention further pertains to novel agents identified by the herein-described screening assays and uses thereof for treatments as described herein, for example, for the enhancement of longevity in an animal, including humans.

In one embodiment the invention provides soluble assays using a cellular modulator of CRTC activity, or a cell or tissue expressing a cellular modulator of CRTC activity, either naturally occurring or recombinant. In another embodiment, the invention provides solid phase based in vitro assays in a high throughput format, where a cellular modulator of CRTC activity is attached to a solid phase substrate via covalent or non-covalent interactions.

“Inhibitors,” “activators,” and “modulators” of a CRTC activity in cells are used to refer to inhibitory, activating, or modulating molecules, respectively, identified using in vitro and in vivo assays for CRTC activity, e.g., ligands, agonists, antagonists, and their homologs and mimetics.

“Modulator” includes inhibitors and activators. Inhibitors are agents that, e.g., bind to, partially or totally block stimulation, decrease, prevent, delay activation, inactivate, desensitize, or down regulate CRTC activity, e.g., antagonists. Activators are agents that, e.g., bind to, stimulate, increase, open, activate, facilitate, enhance activation, sensitize or up regulate a CRTC activity, e.g., agonists. Modulators include genetically modified versions of biological molecules with a CRTC activity, e.g., with altered activity, as well as naturally occurring and synthetic ligands, antagonists, agonists, small chemical molecules and the like.

“Cell-based assays” for inhibitors and activators include, e.g., applying putative modulator compounds to a biological sample having CRTC activity and then determining the functional effects on CRTC activity, as described herein. “Cell based assays” include, but are not limited to, in vivo tissue or cell samples from a mammalian subject or in vitro cell-based assays comprising a biological sample having CRTC activity that are treated with a potential activator, inhibitor, or modulator and are compared to control samples without the inhibitor, activator, or modulator to examine the extent of inhibition.

“Compound” or “test compound” refers to any compound tested as a modulator of CRTC activity. The test compound can be any small organic molecule, or a biological entity, such as a protein, e.g., an antibody or peptide, a sugar, a nucleic acid, e.g., an antisense oligonucleotide, RNAi, or a ribozyme, or a lipid. Alternatively, a test compound can be modulators of biological activities that affect a CRTC activity. Typically, test compounds will be small organic molecules, nucleic acids, peptides, lipids, or lipid analogs.

The invention will now be further described by way of the following non-limiting examples.

### Example 1

### C. Elegans CRTC-1 Mediates Longevity

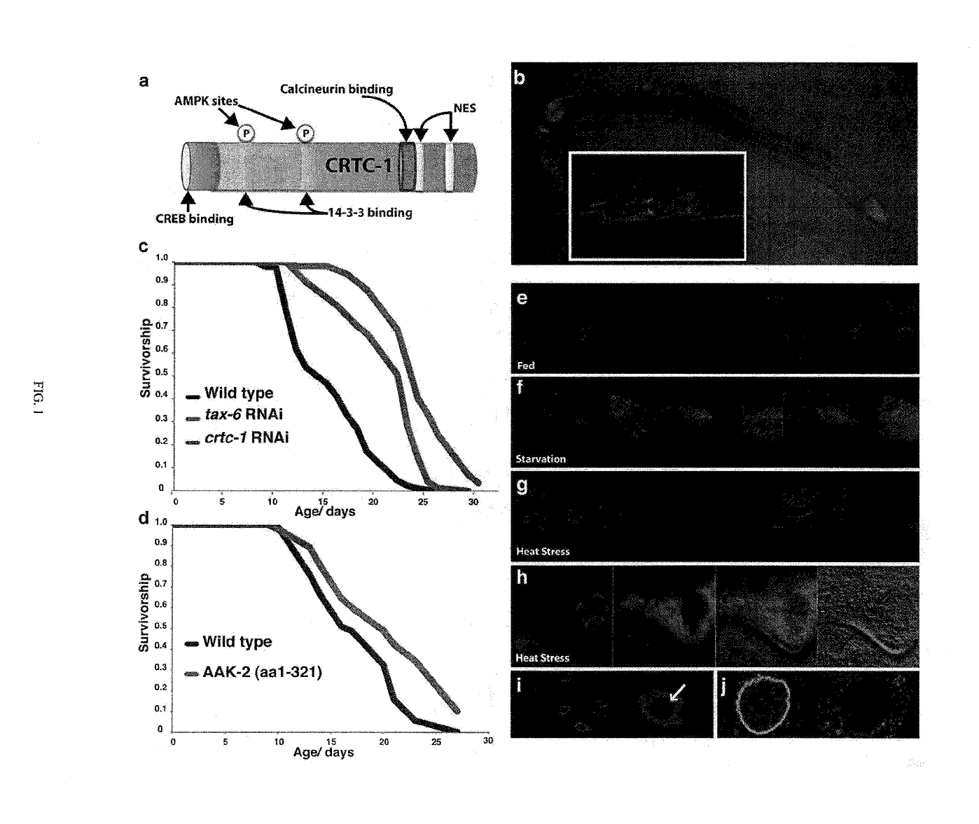

Since both activation of AMPK and inactivation of calcineurin in mammals reduce CRTC activity^{8,10}, and these same conditions extend lifespan in the worm^{6,7,13} Applicants hypothesised that reduction of CRTCs themselves might be sufficient to increase lifespan. Homology search of human CRTC 1 and 2 within the completed C. elegans genome revealed Y20F4.2 as the only CRTC family member in the worm (FIG. 7a). Applicants therefore named Y20F4.2 crtc-1 (FIG. 1a, 7a). Further sequence analysis led to the identification of conserved AMPK phosphorylation and calcineurin binding sites (FIG. 1a, 7a). To determine if crtc-1 was expressed in the same tissues as AMPK and calcineurin, Applicants generated worms expressing red fluorescent protein (RFP) under control of the crtc-1 promoter. crtc-1 is expressed throughout the intestine of the worm, as well as neurons in the head and tail (FIG. 1b), overlapping the expression pattern of the C. elegans calcineurin catalytic A subunit, tax-6⁷ and AMPK catalytic subunit, aak-2 (FIG. 7c). Applicants then engineered a transgenic C. elegans strain expressing recombinant CRTC-1 fused to RFP (CRTC-1::RFP) to monitor CRTC-1 in vivo. RNA interference (RNAi) of crtc-1 by feeding bacteria expressing crtc-1 dsRNA to transgenic worms expressing the CRTC-1::RFP fusion protein ablated its expression through out the intestine (FIG. 7d) but did not deplete expression in neurons due to their refractory nature to RNAi¹⁴. Applicants then tested whether RNAi mediated knockdown of crtc-1 could increase lifespan. Strikingly, and in accordance with the notion that CRTC-1 is central to calcineurin and AMPK regulated longevity, inhibition of crtc-1 via RNAi extended wild type median lifespan by 53% compared to untreated controls (FIG. 1c, Table 2).

### Example 2

### AMPK and Calcineurin Regulate CRTC-1 in Vivo

Mammalian calcineurin activates CRTCs⁸ and studies in C. elegans involving tax-6 mutants have established calcineurin's involvement in lifespan regulation⁷. Applicants reduced tax-6 expression by RNAi and found it recapitulated the lifespan extension seen by crtc-1 knockdown. In wild type worms tax-6 RNAi increased median lifespan by 60% (FIG. 1c), presumably by reducing CRTC-1 function. Since activated AMPK inactivates CRTCs in mammals¹⁰ Applicants generated worms expressing a truncated gain-of-function AMPK catalytic subunit, AAK-2 (aa 1-321), a strategy that in mammalian cells exposes the activation domain of AMPK to activating upstream kinases due to a lack of gamma regulatory subunit binding potentia^{15,16,17}. Worms expressing AAK-2 (aa 1-321) were long-lived and displayed increased stress resistance (FIG. 1d a & 8 respectively).

Because reducing crtc-1 expression resulted in increased longevity similar to reducing tax-6 or activating AAK-2 Applicants asked whether altering AMPK or calcineurin activity regulated CRTC-1 in vivo. Applicants first tested if environmental conditions that extend lifespan by AMPK activation also inactivate CRTC-1. AMPK is activated when energy is low in response to an increase in the AMP/ATP ratio¹⁸, and in C. elegans in response to starvation and heat stress⁶. Nutrient deprivation increases C. elegans lifespan¹, while heat stress can induce a pro-longevity hormetic response in the worm dependent upon aak-2⁶. In mammals, activated AMPK down-regulates CRTC2 by altering its cellular localization, rendering it cytosolic and therefore unable to activate transcription¹⁰. Applicants therefore tested the effect of starvation and heat stress on CRTC-1 localization. Under basal conditions CRTC-1::RFP was present throughout the nucleus and cytosol (FIG. 1e, FIG. 7d). However, both starvation and heat stress induced nuclear exclusion of CRTC-1::RFP and its translocation to the cytosol of intestinal cells (FIG. 1 f & g). Applicants confirmed nuclear exclusion of CRTC-1 after heat stress both by DAPI staining (FIG. 1 h&i) and by creating a transgenic line coexpressing CRTC-1::RFP and a GFP fused NUP160 nuclear pore subunit to identify the nuclear envelope¹⁹ (FIG. 1j).

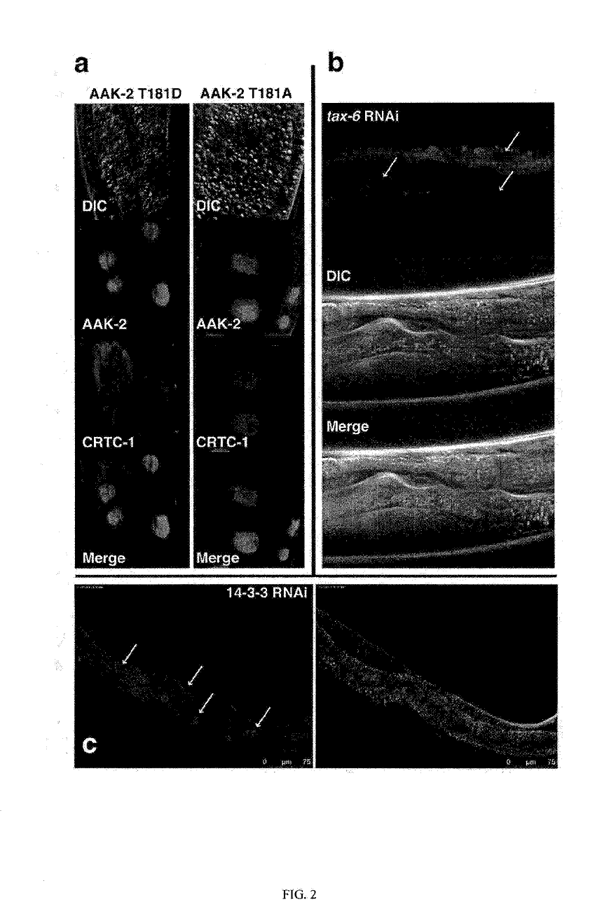

Since environmental conditions that activate AMPK and increase longevity caused CRTC-1 to be retained in the cytoplasm, Applicants tested whether direct activation of AMPK also inactivated CRTC-1 by constructing a constitutively active AAK-2. Mammalian AMPK alpha is active when phosphorylated at threonine 172 in its activation loop and mutation of this residue to aspartic acid (T172D) or alanine (T172A) results in a constitutively active or kinase-dead AMPK respectivelyl¹⁵. Alignment of the C. elegans AAK-2c and mammalian AMPK alpha 2 revealed Thr181 as the conserved activation loop phosphorylation site in worms, allowing Applicants to constitutively activate or inactivate AAK-2 via mutation. Expression of CRTC-1::RFP with active AAK-2c (aa 1-321, T181D)::GFP, resulted in nuclear exclusion of CRTC-1 under well-fed conditions at 20° C. (FIG. 2a), confirming the ability of AMPK to inactivate CRTC-1 in vivo. In contrast, coexpression of kinase dead AAK-2c (aa 1-321, T181A)::GFP and CRTC-1::RFP did not result in nuclear exclusion of CRTC-1 (FIG. 2a), demonstrating that catalytic activation of AAK-2 by Thr181 phosphorylation is important for CRTC-1 nuclear exclusion.

To test if reducing tax-6 inactivated CRTC-1 in vivo, Applicants monitored CRTC-1::RFP localization in worms subjected to tax-6 RNAi. In mammals, calcineurin dephosphorylates CRTC2 resulting in its nuclear localization and activation⁸. Therefore if reducing tax-6 extends lifespan by inactivating CRTC-1 in the worm, tax-6 RNAi would result in CRTC-1 being sequestered to the cytosol and inactivated. In support of this hypothesis and consistent with TAX-6 being the essential phosphatase for CRTC-1 regulation, Applicants found CRTC-1 to be nuclear excluded in tax-6 RNAi treated animals (FIG. 2b).

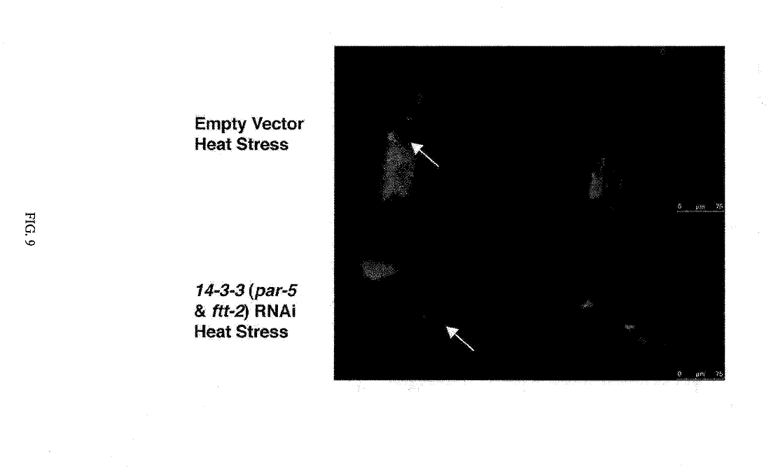

To further elucidate the mechanism by which AMPK and calcineurin regulate CRTC-1 in C. elegans, Applicants tested the effect of depleting 14-3-3 proteins. In mammals phosphorylation of CRTC2 at serine 171 by active AMPK or SIK^{8,10} causes a conformation change that exposes a 14-3-3 protein binding site⁸. 14-3-3 binding retains CRTC2 in the cytoplasm and as such renders it inactive^{8,20}. C. elegans have two 14-3-3 proteins, FTT-2 and PAR-5²¹ and, as expected, simultaneous knock down of both 14-3-3s via RNAi resulted in CRTC-1::RFP accumulation within the nucleus (FIG. 2c). Moreover, environmentally induced CRTC-1 inactivation requires 14-3-3 proteins, since heat stress did not result in CRTC-1::RFP cytosolic sequestering in worms treated with 14-3-3 RNAi (FIG. 9).

### Example 3

### AMPK/Calcineurin Regulate CRTC-1 via Conserved Phosphorylation Sites

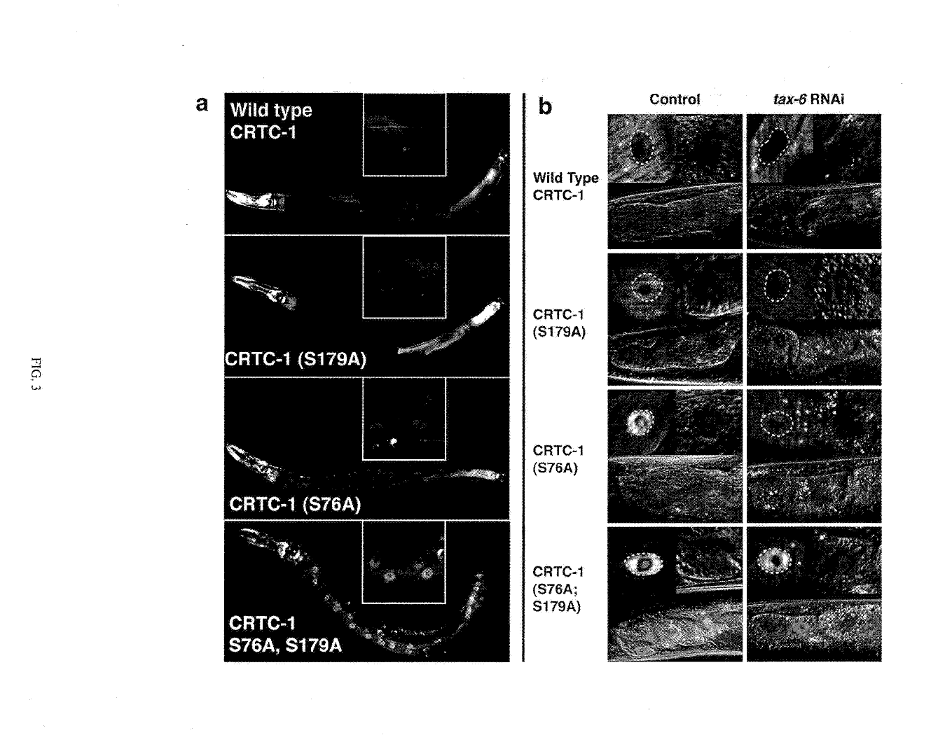

Having determined that the pro-longevity effects of increasing AMPK activity and reducing calcineurin both inactivate CRTC-1, Applicants next tested if they did so by antagonistically regulating the phosphorylation status of CRTC-1. Cellular localization of CRTC2 in mammals is predominantly regulated by SIK/AMPK at Ser171^{8,10}, although phosphorylation by MARK2 at Ser275 has also been shown to play a role²². The conserved worm AMPK/SIK site corresponding to mouse CRTC2 Ser171 is CRTC-1 Ser179, which resides within a 14-3-3 binding domain (FIG. 7 a & b). Surprisingly, mutation of this serine to alanine (S179A) had nominal affect on the cellular localization of CRTC-1 (FIG. 3a). Without being bound to theory, Applicants' believe this suggests that alternative phosphorylation events may be essential for CRTC-1 localization. Another potential AMPK/SIK phosphorylation site within CRTC-1 is Ser76, which also lies inside a 14-3-3 binding domain. By alignment CRTC-1 Ser76 corresponds to the mammalian CRTC2 Ser70, which is phosphorylated by AMPK family members^{23,24}. Mutation of CRTC-1 Ser76 to alanine (S76A) resulted in retention of CRTC-1 in intestinal nuclei (FIG. 3a). Interestingly, combination of the two point mutants in a S76A, S179A double mutant appeared to have an additive effect that further enhanced nuclear localization of CRTC-1 (FIG. 3a).

To determine if TAX-6 altered the phosphorylation status of the conserved AMPK target residues of CRTC-1 Applicants examined the effects of tax-6 RNAi on the cellular localization of the CRTC-1 serine mutants. tax-6 RNAi resulted in complete nuclear exclusion of wild type CRTC-1 (FIG. 3b). Localization of CRTC-1 (S179A) or CRTC-1 (S76A) showed sensitivity to tax-6 RNAi (FIG. 3b). Strikingly, nuclear exclusion via tax-6 RNAi was completely blocked in the double mutant CRTC-1 (S76A, S179A)::RFP transgenic line, in which CRTC-1 is constitutively nuclear in both control and tax-6 RNAi conditions (FIG. 3b). Taken together these findings establish that AAK-2 and TAX-6 antagonistically regulate CRTC-1 activity in vivo through post-translational modification of identical serine residues, reinforcing CRTC-1 as a shared longevity target of AMPK and calcineurin.

### Example 4

### Pharmacological Activation of CRTC-1

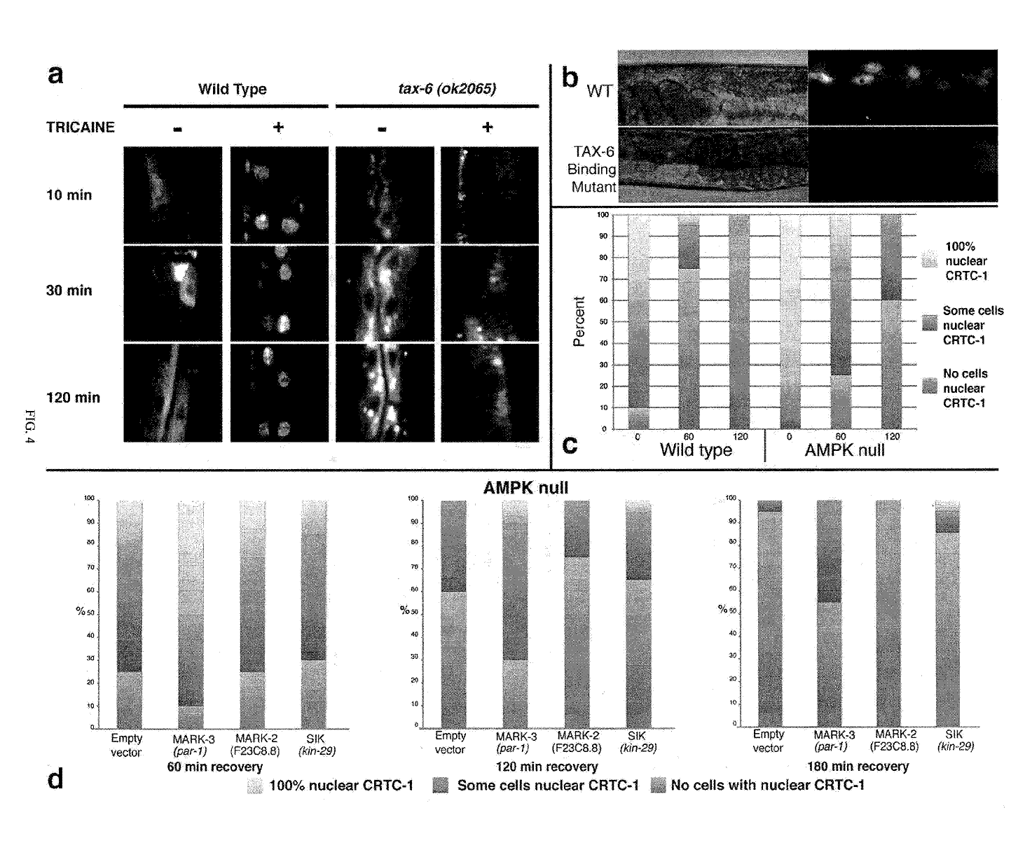

Because environmental cues that inactivate CRTC-1 function through calcineurin and AMPK, Applicants tested whether external stimuli that activate CRTC-1 function through this shared molecular mechanism. Water-soluble local anaesthetics (LAs), such as lidocaine, agonize transient receptor potential (TRP) channel family members and as such increase calcium flux into sensory neurons²⁵. Since calcineurin activity is increased in response to calcium levels²⁶ and overexpression of TRP channels in HeLa cells induces CRTC nuclear translocation²⁰, Applicants tested whether LAs could affect CRTC-1 localization via pharmacological activation of calcineurin. Tricaine is a LA commonly used to anesthetize C. elegans that is structurally similar to lidocaine. Tricaine administration resulted in the rapid nuclear localization of CRTC-1 in C. elegans intestinal cells (FIG. 4a). Significantly, this effect was completely dependent on the presence of TAX-6. Without being bound to theory, Applicants believe this supports their belief that calcineurin is the key phosphatase regulating CRTC-1 activity in the worm. When expressed in a tax-6 (ok2065) deletion mutant, CRTC-1::RFP was invariably cytosolic under basal conditions and after 2 hours of tricaine treatment (FIG. 4a). Furthermore, CRTC-1::RFP containing site-specific mutations in the calcineurin binding site did not shuttle into the nucleus in response to tricaine treatment (FIG. 4b).

### Example 5

### Redundancy of AMPK Family Members for CRTC-1 Regulation

Applicants took advantage of the fact that the nuclear localization of CRTC-1 following tricaine administration was reversible within two hours of drug removal to investigate whether AMPK was the sole kinase regulating CRTC-1 (FIG. 4c). Since activated AMPK drove CRTC-1 into the cytosol (FIG. 2a) Applicants tested whether AMPK was required for the cytosolic relocalization after tricaine was removed. However, although the tricaine-induced nuclear localization of CRTC-1 was expedited in an AMPK null mutant (FIG. 4c), upon removal of the drug, CRTC-1 relocated to the cytoplasm despite the absence of AMPK. Accordingly, in certain embodiments, additional kinases besides AMPK may play a role in CRTC-1 regulation.

AMPK/LKB-1 family kinases are highly conserved between mammals and the worm (Table 3), and in mammals multiple AMPK kinases can phosphorylate CRTCs^{8,10,22}. To test for functional redundancy between these kinases, Applicants looked for additive effects on the rate at which CRTC-1 returned to the cytosol after removal of tricaine in AMPK null worms subjected to RNAi of other AMPK family members. As would be expected if CRTC-1 were a target of more than one AMPK family kinase, Applicants found that RNAi of other kinases, in particular the MARK3 orthologue par-1, further expedited nuclear localization of CRTC-1 upon tricaine treatment and delayed its subsequent cytosolic relocation post drug removal in worms lacking AMPK (FIG. 4d). Interestingly, unlike other multicellular organisms in which AMPK is critical for embryonic development^{27,28}, C. elegans can survive and become viable adults without AMPK^{29,30}, supporting the notion of functional redundancy between AMPK family kinases in the worm. This redundancy may also explain observations that AMPK is required for lifespan extension in C. elegans by some dietary restriction protocols¹³ but not others²⁹. Although AMPK is activated under low nutrient conditions in wild type animals, the pro-longevity role of AMPK in dietary restriction may be compensated for by similar kinases in AMPK null animals subjected to diet restriction²⁹.

### Example 6

### Calcineurin Mediates Longevity Via CRTC-1

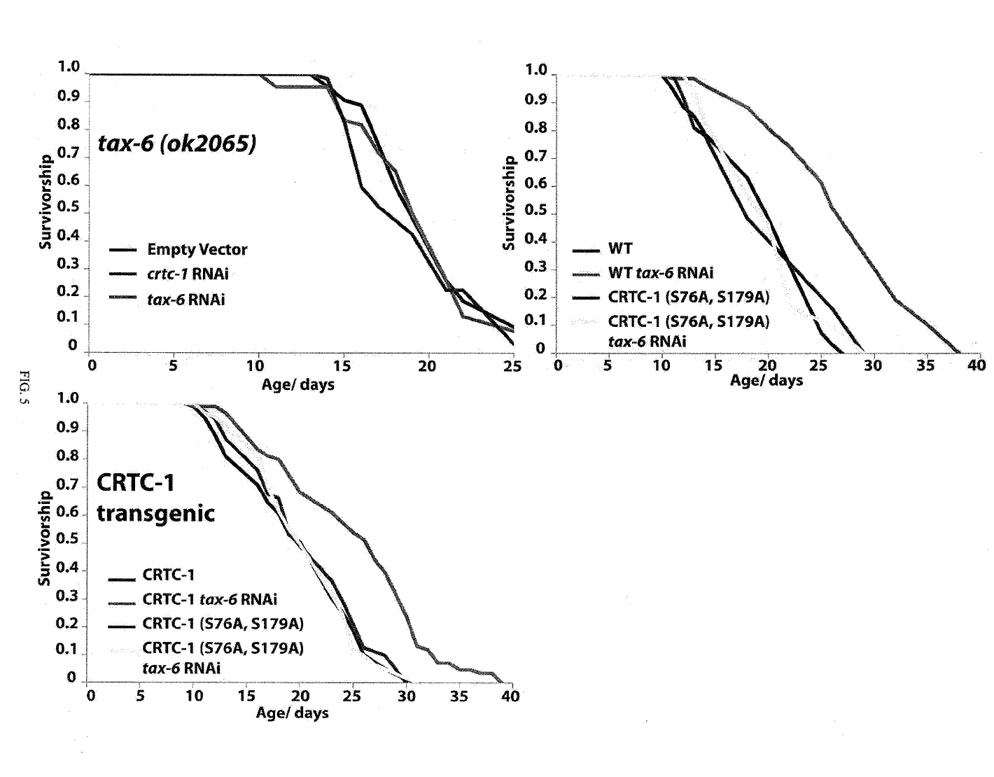

As TAX-6 is important in the regulation of CRTC-1 activity (FIG. 2b, 3b, 4a), Applicants tested if the longevity effects of reducing calcineurin functioned exclusively through inactivation of CRTC-1. CRTC-1 is rendered inactive when tax-6 is reduced, and accordingly the already extended lifespan of tax-6 mutants was not further increased by crtc-1 RNAi (FIG. 5a, Table 2).

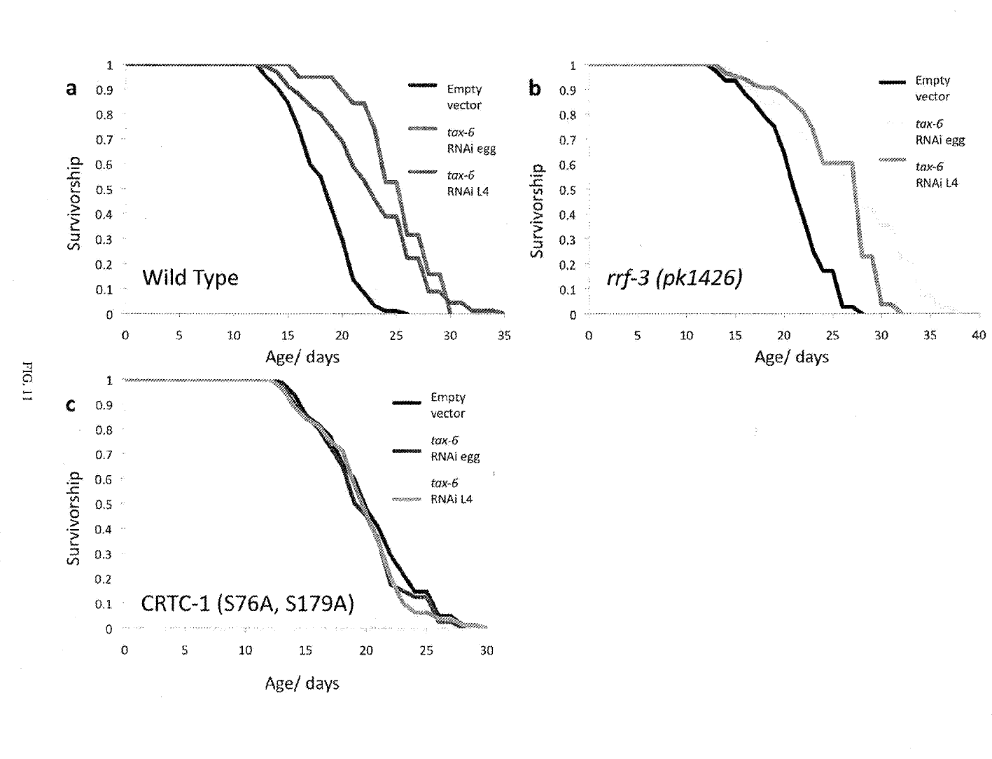

To further determine whether calcineurin mediated lifespan via CRTC-1 Applicants tested the effect of tax-6 RNAi on worms expressing either wild type or constitutively active CRTC-1. Applicants reasoned that if C. elegans subjected to tax-6 RNAi are long lived due to inactivation of CRTC-1, reducing tax-6 should have no effect on lifespan in worms expressing the constitutively active nuclear CRTC-1 (S76A, S179A). Consistent with this notion, tax-6 RNAi robustly extended the lifespan of wild type worms (FIG. 5b, Table 2), yet had no effect on worms expressing constitutively nuclear CRTC-1 (S76A, S179A) (FIG. 5b, Table 2). This suppression was not due to general sickness or developmental effects as there was no significant difference between the lifespan of wild type worms and those expressing CRTC-1 (S76A, S179A) (FIG. 5b, Table 2). Also, knocking down tax-6 specifically during adulthood still increased wild type lifespan but had no effect on the CRTC-1 (S76A, S179A) phospho-mutant (FIG. 11). Interestingly, the CRTC-1 (S76A, S179A) suppression of tax-6 RNAi mediated lifespan extension was dependent upon the nuclear localization of CRTC-1, since lifespan was increased by tax-6 RNAi in worms expressing wild type CRTC-1::RFP, which translocates to the cytoplasm freely when calcineurin is not present (FIG. 5c, Table 2).

### Example 7

### CRTC-1 Modulates CREB Homologue 1 (CRH-1) to Regulate Longevity

Nutritional conditions that increase lifespan inactivate CRTC-1. Consequently Applicants examined whether environmental regulation of longevity via this pathway acts through downstream changes in energy homeostasis. CRTCs are well established transcriptional coactivators of the transcription factor CREB. CRTCs bind as tetramers to the bZIP domain of CREB and facilitate recruitment of the transcriptional apparatus⁹. CREB dependent transcription is involved in a diversity of key processes in mammals including memory, immune function, DNA repair, and in particular energy homeostasis and fat storage³¹. Unlike mammals, which possess three CREB family members, CREB, CREM and ATF-1³¹, CRH-1 is the sole C. elegans CREB orthologue³². To determine if the effects of AMPK, TAX-6 and CRTC-1 on longevity function via CRH-1, Applicants first examined the interaction between CRTC-1 and CRH-1. Previously, in situ hybridisation of crh-1 mRNA in adult worms reported expression of crh-1 in neurons and the gonad³². However, as CREB is ubiquitously expressed in mammals, Applicants further examined the expression pattern of crh-1 using a reporter construct containing the promoter region of the largest crh-1 isoform, crh-1d, driving GFP. Expression of crh-1 was seen ubiquitously throughout the worm and in overlapping tissues to crtc-1 (FIG. 6a). After confirming the spatiotemporal requirements for interaction were fulfilled, Applicants identified that Arg314, the critical residue in CREB needed to complex with CRTCs in mammals, was conserved in CRH-1 at Arg276. To explore the potential interaction of these proteins Applicants performed immunoprecipitation studies in 293T cells. Communoprecipitation of FLAG-tagged CRTC-1 and HA-tagged CRH-1 indicated that these proteins interact in vivo (FIG. 6b). Incubation with excess FLAG peptide in parallel confirmed that CRH-1 enrichment was specific to FLAG-tagged CRTC-1.

Since CRTC-1 bound to CRH-1 in vivo, and because reduction of crtc-1 extended lifespan, Applicants next tested whether reduction of crh-1 expression might also increase longevity. RNAi of crh-1 did not increase the lifespan of wild type worms but did result in significant lifespan extension of rrf-3 (pk1426) mutants (FIG. 6c, Table 2), which display increased sensitivity to RNAi and allow more efficient gene knockdown in neurons than is achievable in wild type worms¹⁴. Lifespan extension via crh-1 RNAi was not as strong as that seen by tax-6 or crtc-1 RNAi and was greater when applied only in adulthood rather than throughout development (Table 2). CREB regulates transcription in both a CRTC dependent and independent manner, consequently RNAi for crh-1 may reduce expression of non-CRTC-1 dependent targets that have critical roles unrelated to longevity. Applicants therefore focused specifically on CREB targets involved in energy homeostasis, since AMPK and calcineurin are nutrient responsive, and CRTC-1 is inactivated under low energy conditions in the worm.

### Example 8

### Energy Homeostasis Targets of CRTC-1 and CRH-1 Regulate Longevity

In mammals, CREB regulates energy homeostasis in the liver, in particular by increasing glucose production in response to fasting via activation of gluconeogenic enzymes such as the phosphoenol pyruvate carboxykinases (PEPCKs) and G6 Pase in a CRTC dependent manner¹⁰. CREB binds with high affinity to the cAMP responsive element (CRE) to mediate transcriptional response of target genes⁹. Unlike mammals, which show positional bias for the CRE, invertebrates can have functional CREs thousands of bases upstream of the transcriptional start site³³. Analysis of the 2 kb region 5′ to the transcriptional start site revealed CREs in the C. elegans PEPCK orthologues R11A5.4 and H04M03.1. Supporting Applicants' belief that environmental conditions can modulate energy homeostasis to increase lifespan via a conserved signalling pathway, RNAi of either R11A5.4 or H04M03.1 extended lifespan (FIG. 6d). In addition to regulating gluconeogenesis, PEPCKs also function in glycolysis, carbon dioxide fixation and glyceroneogenesis³⁴. If PEPCKs affect ageing via their gluconeogenic role, RNAi for other gluconeogenic genes might phenocopy their longevity effects. Applicants examined the effect on lifespan of perturbation to other genes in the C. elegans gluconeogenic Gene Ontology (GO) category. Along with R11A5.4 and H04M03.1, the worm gluconeogenic GO group comprises a third PEPCK (W05G11.6), the pyruvate carboxylase orthologue pyc-1 and the glucose 6-phosphate isomerase (gpi-1). RNAi of each gene in the gluconeogenic GO list increased lifespan of wild type animals (FIG. 6d). Hence at least one downstream node of CRTC-1 and CRH-1 that regulates lifespan is the gluconeogenic module of energy homeostasis.

### Example 9

### Materials and Methods

C. elegans strains, growth, imaging, lifespan analysis and RNAi application were performed as previously described⁵⁴. Transgenic strains were generated via microinjection into the gonad of adult hermaphrodites using standard techniques. Integrated transgenic lines were generated using gamma irradiation and out-crossed to wild type at least four times. All lifespans were conducted at 20° C. with deaths scored and live worms transferred to new plates every 1-2 days. JMP 8 was used for all statistical analysis.

Lifespan Studies

All lifespan experiments were performed on standard 6 cm nematode growth media plates (Hope, I. A. (ed B. D. Hames) (Oxford University Press, New York, 1999)) supplemented with 100 μg/ml carbenicillin at 20° C. Plates were removed from 4° C. storage 2 days before seeding with 100 μl of E. coli HT115 containing either empty vector plasmid or RNAi inducing plasmids. RNA interference (RNAi) for a particular gene can be readily achieved in the worm by feeding C. elegans E. coli (HT115) that express double stranded RNA of the gene of interest (Fire, A. et al. Potent and specific genetic interference by double-stranded RNA in Caenorhabditis elegans. Nature 391, 806-811, doi:10.1038/35888 (1998)). Bacterial cultures were grown overnight at 37° C. under the presence of both carbenicillin (100 μg/ml) and tetracycline (10 μg/ml) selection before seeding onto NGM plates. Once seeded, bacterial lawns were grown at room temperature for 24 hours. RNAi was induced with 100 μl IPTG (100 mM) 2 hours before worms were added to plates. To age-synchronize worms, 5 gravid adults (24 hours post L4) were placed on plates with the appropriate control or RNAi bacteria and allowed to lay eggs for 5 hours before being removed. These eggs were then cultured to adulthood (72 hours post egg lay at 20° C.) before being moved to fresh plates at a density of 10 worms per plate, 10 plates per treatment. Age=0 was day adults were moved to 10 worms a plate. Worms were moved to fresh plates every 1-2 days until day 14, after which only worms on mold contaminated plates were transferred. Worms were censored at the first sign of any bacterial contamination. Death was scored by gentle agitation with a worm pick and confirmed with no response after three attempts at both the head and tail. Death was scored every 1-2 days throughout.

RNAi Constructs

With exception of crtc-1, all RNAi constructs used were taken from either the Vidal or Ahringer RNAi libraries. crtc-1 RNAi plasmid was made by cloning full length crtc-1 cDNA between the two inverted T7 promoters in the pAD1 RNAi plasmid and transforming into HT115 cells.

DAPI Staining

Worms were washed in 1 ml M9 (Hope, I. A. (ed B. D. Hames) (Oxford University Press, New York, 1999)) in watch glass (1-3×, until bacteria is removed). M9 was almost completely removed before 100-200 μl DAPI (200 ng/ml in EtOH) was added. DAPI treatment was left to sit in dark for about 20 minutes or until Ethanol is evaporated. 1 ml M9 was then added to rehydrate for 1 hour (up to 5 hours at RT or O/N at 4° C.). One drop (˜3 μl) prolong mounting media was placed into on a slide before transferring stained worm. A cover slip was added and sealed with nail polish. Fluorescence was examined at 358 nm.

Heat Stress/Starvation Assays

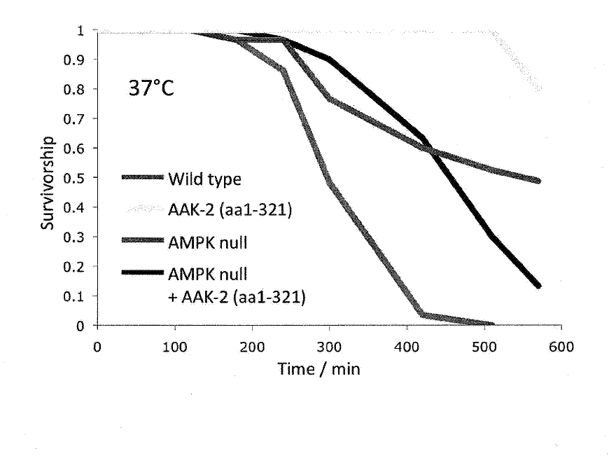

Effects of heat and starvation on CRTC::RFP was measured by placing worms expressing crtc-1::RFP onto either OP50 seeded NGM plates at 33° C., O/N or into 9 well plates containing M9 media at 20° C. Controls were worms on OP50 at 20° C. Survival under heat stress in FIG. 8 was performed at 37° C.

Microscopy

All microscopy was performed using 0.1 mg/ml tetramisole hydrochloride in M9 as an anesthetic, which pilot experiments revealed had no effect on CRTC-1 localization. Except for tricaine time course experiments, worms were in 5 μl anesthetic mounted on 2% agarose pads on glass slides under glass cover slips. All photos were taken using a Zeiss Axiovert microscope & Axiocam. Pictures in FIGS. 2a and 3b used apotome optical sectioning. For the tricaine experiments (FIG. 4a&b), L4 worms were placed in wells of a 96 well plate in 100 μl of tetramisole/M9 with or without tricaine (2 mg/ml). Pictures were taken during time course through the 96 well plate.

Kinase Redundancy Assays

Worms were subjected to RNAi for AMPK family kinases from hatch. 24 hours post L4 worms were then picked into M9 with tricaine (2 mg/ml) in wells of a 9 well plate. Worms were left on a rotational shaker at 20° C. for 2 hours. Using a glass pipette (Mair, W. A simple yet effective method to manipulate C. elegans in liquid. Worm Breeders Gazette 18 (2009)), worms were then placed onto NG plates seeded with E. coli (0P50). When tricaine solution had evaporated (approx 20 min), worms were picked onto fresh OP50 plates, 5 worms per plate. Localization of CRTC::RFP was then scored as ‘All nuclear’ (all intestinal cells showed only punctate nuclear CRTC-1), ‘Some cells nuclear’ (intestinal cells showed mix of punctate nuclear CRTC and cytosolic CRTC) and ‘No cells nuclear’ (CRTC was dispersed evenly throughout nucleus and cytosol in all intestinal cells). Time=0 was when moved to fresh OP50 plates.

Transgenic Strain Construction

Expression constructs were based upon the pPD95.77 from the Fire lab C. elegans vector kit. RFP in manuscript refers to tdTOMATO, which replaced the GFP in pPD95.77. Transgenic strains were generated via microinjection into the gonad of adult hermaphrodites using standard techniques. Integrated transgenic lines were generated using gamma irradiation and out-crossed to wild type at least four times.

Calcineurin Binding Mutant

Quik change mutagenesis was used to mutate residues within the conserved calcineurin binding site in CRTC-1. This resulted in changing the amino acid sequence (aa423-428) as follows:

WT: EALDIPKLTITNAEGA

Calcineurin binding mutant: EALDIAKATAANAEGA

TABLE 1
Strains used.

| Strain | Genotype | Description | Figure |
|---|---|---|---|
| AGD534 | N2, UthEx[CRTC-1p::RFP, rol-6] | CRTC-1 promoter driving RFP. | 1b, 6a, |
| AGD445 | N2, UthEx201[crtc-1p::crtc-1::RFP::unc-54 3′UTR, | CRTC-1::RFP fusion combined | 1e-g, 1j |
| | NUP160p::NUP160::GFP, rol6] | with NUP160::GFP fusion | |
| AGD383 | N2; uthls202 [aak-2cp:: aak2 (aa1-321)::Tomato::unc-54 | Integrated AAK-2 GOF | 1d |
| | 3′UTR, rol6] | | |
| AGD467 | N2, UthEx[aak-2 intron 1::aak2(aa1-321 T172D)::GFP; | AAK-2 CA::GFP and CRTC- | 2a |
| | crtc-1p::crtc-1::RFP; rol6] | 1::RFP | |
| AGD469 | N2, UthEx[aak-2 intron 1::aak2(aa1-321 T172A)::GFP; | AAK-2 kinase dead::GFP and | 2a |
| | crtc-1p::crtc-1::RFP; rol6] | CRTC-1::RFP | |
| AGD418 | N2; uthls205 [crtc-1p::crtc-1::RFP::unc-54 3′UTR, rol-6] | Integrated CRTC-1::RFP fusion | 2b&c, |
| | | | 3a&b, 4a, |
| | | | 4b, 4c, 5c, |
| | | | 7d, 9 |
| AGD448 | N2, UthEx[crtc-1p::crtc-1 (S179A)::RFP; rol6] | CRTC-1 S179A mutant | 3a&b |
| AGD470 | N2, UthEx[crtc-1p::crtc-1 (S76A)::RFP; rol6] | CRTC-1 S76A mutant | 3a&b |
| AGD466 | N2, uthEX222 [crtc-1p::crtc-1 (S76A, S179A)::RFP::unc-54 | CRTC-1 S76A, S179A mutant | 3a&b, |
| | 3′UTR, rol6] | | 5b&c |
| AGD426 | tax-6 (ok2065) IV, uthls205 [crtc-1p::crtc-1::RFP::unc-54 | Integrated CRTC-1::RFP fusion | 4a |
| | 3′UTR, rol-6] | in tax-6 null. | |
| AGD553 | N2, uthEx[crtc-1p::crtc-1 (cal mutant)::RFP::unc54 3′UTR, | CRTC-1 with calcineurin binding | 4b |
| | rol-6] | site mutated | |
| AGD397 | aak-1 (tm1944) III; aak-2 (ok524) X, uthEx202[crtc-1p::crtc- | CRTC-1::RFP in AMPK null | 4c&d |
| | 1::RFP::unc-54 3′UTR, rol6] | | |
| N2 | +/+ | Wild type | 1c&d, 5b, |
| | | | 6d, 8, 10 |
| RB1667 | tax-6(ok2065) IV | Tax-6 null | 5a |
| AGD472 | N2, uthEx[crh-1p::GFP::unc-54 UTR, rol-6] | Crh-1 promoter driving GFP | 6a |
| NL2099 | rrf-3 (pk1426) II | rrf-3 RNAi sensitive mutant | 6c |
| AGD444 | aak-1 (tm1944) III; aak-2 (ok524) X | AMPK null | 8 |
| AGD587 | N2; uthEX [aak-2cp:: aak2 (aa1-321)::Tomato::unc-54 | AAK-2 GOF | 8 |
| | 3′UTR, rol6] | | |
| AGD591 | aak-1 (tm1944) III; aak-2 (ok524) X, uthEx [aak-2cp:: aak2 | AAK-2 GOF in AMPK null | 8 |
| | (aa1-321)::Tomato::unc-54 3′UTR, rol6] | | |

Single Worm PCR for Genotyping

Single Worm Lysis Buffer: 30 mM Tris pH8.0, 8 mM EDTA, 100 mM NaCl, 0.7% NP-40, 0.7% Tween-20. Add PK to the final concentration of 100 μg/ml just before use. Preparation of DNA template. Add one worm to a PCR tube containing 5 μl SWLB supplemented with PK. Incubate 60 min at 60° C. Heat inactivate PK at 95° C. for 15 min. Cool reaction to 4° C. Setup of PCR reaction: Use 50 of the worm lysate as template. Setup PCR reaction as appropriate.

Expression of Worm Proteins in 293T Cells

Full length C. elegans crtc-1 and crh-1 cDNA was cloned using the gateway recombination into mammalian expression destination plasmids pcDNA3 containing in frame 5′ FLAG and HA tags respectively. 293T cells were transfected using lipofectamine using standard techniques.

Immunoprecipitation

2 10 cm plates of 293T per sample. Washed 1× with 10 ml PBS and re-suspended in 500 μl PLB. Incubated 10′ at 4° C. Sonicated 6×10″ on 15″ off output 4. Rest on ice for 2′ and repeat. Cleared 10′ 4° C. at 10,000 g. Added 20 μl p.v prot G/A to each at 4° C. for one hour. Centrifuged to clear. Pooled together and divided equally for each treatment. Used 1.1 ml cleared lysate for each. Added 25 μl p.v FLAG or HA beads. For controls pre-blocked with FLAG and/or HA 25 μg for 1 hour at 4° C. on ice then added 25 μg more during immunoprecipitation (50 μg total peptide). Incubated immunoprecipitates O/N at 4° C. with rotation. Washed 4× with 1 ml PLB buffer. Eluted with HA or FLAG peptide 1 mg/ml (32 μl) RT for 30′. Collected 5′ 4000 rpm (30 μl). For Western: HA & FLAG onput—28 μl+7 μl 5×RSB, IPs—28 μl+7 μl 5×RSB, Boiled IP beads—30 μl 2×RSB.

### REFERENCES

1 Mair, W. & Dillin, A. Aging and survival: the genetics of life span extension by dietary restriction. Annu Rev Biochem 77, 727-754, (2008).
2 Wolff, S. & Dillin, A. The trifecta of aging in Caenorhabditis elegans. Exp Gerontol 41, 894-903, (2006).
3 Cohen, E. & Dillin, A. The insulin paradox: aging, proteotoxicity and neurodegeneration. Nat Rev Neurosci 9, 759-767, (2008).
4 Selman, C. et al. Ribosomal protein S6 kinase 1 signaling regulates mammalian life span. Science 326, 140-144, (2009).
5 Stanfel, M. N., Shamieh, L. S., Kaeberlein, M. & Kennedy, B. K. The TOR pathway comes of age. Biochim Biophys Acta 1790, 1067-1074, (2009).
6 Apfeld, J., O'Connor, G., McDonagh, T., DiStefano, P. S. & Curtis, R. The AMP-activated protein kinase AAK-2 links energy levels and insulin-like signals to lifespan in C. elegans. Genes Dev 18, 3004-3009, (2004).
7 Dong, M. Q. et al. Quantitative mass spectrometry identifies insulin signaling targets in C. elegans. Science 317, 660-663, (2007).
8 Screaton, R. A. et al. The CREB coactivator TORC2 functions as a calcium- and cAMP-sensitive coincidence detector. Cell 119, 61-74, (2004).
9 Conkright, M. D. et al. TORCs: transducers of regulated CREB activity. Mol Cell 12, 413-423, (2003).
10 Koo, S. H. et al. The CREB coactivator TORC2 is a key regulator of fasting glucose metabolism. Nature 437, 1109-1111, (2005).
11 Liu, Y. et al. A fasting inducible switch modulates gluconeogenesis via activator/coactivator exchange. Nature 456, 269-273, (2008).
12 Wang, Y., Vera, L., Fischer, W. H. & Montminy, M. The CREB coactivator CRTC2 links hepatic ER stress and fasting gluconeogenesis. Nature 460, 534-537, (2009).
13 Greer, E. L. et al. An AMPK-FOXO pathway mediates longevity induced by a novel method of dietary restriction in C. elegans. Curr Biol 17, 1646-1656, (2007).
14 Simmer, F. et al. Loss of the putative RNA-directed RNA polymerase RRF-3 makes C. elegans hypersensitive to RNAi. Curr Biol 12, 1317-1319, (2002).
15 Crute, B. E., Seefeld, K., Gamble, J., Kemp, B. E. & Witters, L. A. Functional domains of the alpha1 catalytic subunit of the AMP-activated protein kinase. J Biol Chem 273, 35347-35354, (1998).
16 Foretz, M. et al. Short-term overexpression of a constitutively active form of AMP-activated protein kinase in the liver leads to mild hypoglycemia and fatty liver. Diabetes 54, 1331-1339, (2005).
17 Lamia, K. A. et al. AMPK regulates the circadian clock by cryptochrome phosphorylation and degradation. Science 326, 437-440, (2009).
18 Shackelford, D. B. & Shaw, R. J. The LKB1-AMPK pathway: metabolism and growth control in tumour suppression. Nat Rev Cancer 9, 563-575, (2009).
19 D'Angelo, M. A., Raices, M., Panowski, S. H. & Hetzer, M. W. Age-dependent deterioration of nuclear pore complexes causes a loss of nuclear integrity in postmitotic cells. Cell 136, 284-295, (2009).
20 Bittinger, M. A. et al. Activation of cAMP response element-mediated gene expression by regulated nuclear transport of TORC proteins. Curr Biol 14, 2156-2161, (2004).
21 Wang, W. & Shakes, D. C. Expression patterns and transcript processing of ftt-1 and ftt-2, two C. elegans 14-3-3 homologues. J Mol Biol 268, 619-630, (1997).
22 Jansson, D. et al. Glucose controls CREB activity in islet cells via regulated phosphorylation of TORC2. Proc Natl Acad Sci USA 105, 10161-10166, (2008).
23 Al-Hakim, A. K. et al. 14-3-3 cooperates with LKB1 to regulate the activity and localization of QSK and SIK. J Cell Sci 118, 5661-5673, (2005). Dentin, R., Hedrick, S., Xie, J., Yates, J., 3rd & Montminy, M. Hepatic glucose sensing via the CREB coactivator CRTC2. Science 319, 1402-1405, (2008).
25 Leffler, A. et al. The vanilloid receptor TRPV1 is activated and sensitized by local anesthetics in rodent sensory neurons. J Clin Invest 118, 763-776, (2008).
26 Bandyopadhyay, J., Lee, J. & Bandyopadhyay, A. Regulation of calcineurin, a calcium/calmodulin-dependent protein phosphatase, in C. elegans. Mol Cells 18, 10-16, (2004).
27 Lee, J. H. et al. Energy-dependent regulation of cell structure by AMP-activated protein kinase. Nature 447, 1017-1020, (2007).
28 Jorgensen, S. B. et al. Knockout of the alpha1 but not alpha1 5′-AMP-activated protein kinase isoform abolishes 5-aminoimidazole-4-carboxamide-1-beta-4-ribofuranosidebut not contraction-induced glucose uptake in skeletal muscle. J Biol Chem 279, 1070-1079, (2004).
29 Mair, W., Panowski, S. H., Shaw, R. J. & Dillin, A. Optimizing dietary restriction for genetic epistasis analysis and gene discovery in C. elegans. PLoS One 4, e4535, (2009).
30 Narbonne, P. & Roy, R. Caenorhabditis elegans dauers need LKB1/AMPK to ration lipid reserves and ensure long-term survival. Nature 457, 210-214, (2009).
31 Mayr, B. & Montminy, M. Transcriptional regulation by the phosphorylation-dependent factor CREB. Nat Rev Mol Cell Biol 2, 599-609, (2001).
32 Kimura, Y. et al. A CaMK cascade activates CRE-mediated transcription in neurons of Caenorhabditis elegans. EMBO Rep 3, 962-966, (2002).
33 Smith, B. et al. Evolution of motif variants and positional bias of the cyclic-AMP response element. BMC Evol Biol 7 Suppl 1, S15, (2007).
34 Braeckman, B. P., Houthoofd, K. & Vanfleteren, J. R. Intermediary metabolism. WormBook, 1-24, (2009).
35 Erion, D. M. et al. Prevention of hepatic steatosis and hepatic insulin resistance by knockdown of cAMP response element-binding protein. Cell Metab 10, 499-506, (2009).
36 Shaw, R. J. et al. The kinase LKB1 mediates glucose homeostasis in liver and therapeutic effects of metformin. Science 310, 1642-1646, (2005).
37 Altarejos, J. Y. et al. The Creb1 coactivator Crtc1 is required for energy balance and fertility. Nat Med 14, 1112-1117, (2008).
38 Lerner, R. G., Depatie, C., Rutter, G. A., Screaton, R. A. & Balthasar, N. A role for the CREB co-activator CRTC2 in the hypothalamic mechanisms linking glucose sensing with gene regulation. EMBO Rep 10, 1175-1181, (2009).
39 Iourgenko, V. et al. Identification of a family of cAMP response element-binding protein coactivators by genome-scale functional analysis in mammalian cells. Proc Natl Acad Sci USA 100, 12147-12152, (2003).
40 Canettieri, G. et al. The coactivator CRTC1 promotes cell proliferation and transformation via AP-1. Proc Natl Acad Sci USA 106, 1445-1450, (2009).
41 Mauvezin, C. et al. The nuclear cofactor DOR regulates autophagy in mammalian and Drosophila cells. EMBO Rep, (2009).
42 Green, D. R. & Kroemer, G. Cytoplasmic functions of the tumour suppressor p53. Nature 458, 1127-1130, (2009).
43 Tasdemir, E. et al. Regulation of autophagy by cytoplasmic p53. Nat Cell Biol 10, 676-687, (2008).
44 Dhahbi, J. M. et al. Caloric restriction alters the feeding response of key metabolic enzyme genes. Mech Ageing Dev 122, 1033-1048, (2001).
45 Hagopian, K., Ramsey, J. J. & Weindruch, R. Caloric restriction increases gluconeogenic and transaminase enzyme activities in mouse liver. Exp Gerontol 38, 267-278, (2003).
46 McElwee, J. J., Schuster, E., Blanc, E., Thornton, J. & Gems, D. Diapause-associated metabolic traits reiterated in long-lived daf-2 mutants in the nematode Caenorhabditis elegans. Mech Ageing Dev 127, 458-472, (2006).
47 Castelein, N., Hoogewijs, D., De Vreese, A., Braeckman, B. P. & Vanfleteren, J. R. Dietary restriction by growth in axenic medium induces discrete changes in the transcriptional output of genes involved in energy metabolism in Caenorhabditis elegans. Biotechnol J 3, 803-812, (2008).
48 Van Gilst, M. R., Hadjivassiliou, H. & Yamamoto, K. R. A Caenorhabditis elegans nutrient response system partially dependent on nuclear receptor NHR-49. Proc Natl Acad Sci USA 102, 13496-13501, (2005).
49 Hansen, M., Hsu, A. L., Dillin, A. & Kenyon, C. New Genes Tied to Endocrine, Metabolic, and Dietary Regulation of Lifespan from a Caenorhabditis elegans Genomic RNAi Screen. PLoS Genet. 1, e17, (2005).
50 Lee, S. J., Murphy, C. T. & Kenyon, C. Glucose shortens the life span of C. elegans by downregulating DAF-16/FOXO activity and aquaporin gene expression. Cell Metab 10, 379-391, (2009).
51 Schulz, T. J. et al. Glucose restriction extends Caenorhabditis elegans life span by inducing mitochondrial respiration and increasing oxidative stress. Cell Metab 6, 280-293, (2007).
52 Wolff, S. et al. SMK-1, an essential regulator of DAF-16-mediated longevity. Cell 124, 1039-1053, (2006).
53 Copeland, J. M. et al. Extension of Drosophila life span by RNAi of the mitochondrial respiratory chain. Curr Biol 19, 1591-1598, doi:S0960-9822(09)01586-3 [pii] 10.1016/j.cub.2009.08.016 (2009).
54 Dillin, A. et al. Rates of behavior and aging specified by mitochondrial function during development. Science 298, 2398-2401, doi:10.1126/science.1077780 1077780 [pii] (2002).
55 Libert, S. & Pletcher, S. D. Modulation of longevity by environmental sensing. Cell 131, 1231-1234, doi:S0092-8674(07)01598-X [pii] 10.1016/j.cell.2007.12.002 (2007).
56 Spiegelman, B. M. & Heinrich, R. Biological control through regulated transcriptional coactivators. Cell 119, 157-167, doi:S0092867404009456 [pii] 10.1016/j.cell.2004.09.037 (2004).
57 Cheng, A. & Saltiel, A. R. More TORC for the gluconeogenic engine. Bioessays 28, 231-234, doi:10.1002/bies.20375 (2006).
58 Conkright, M. D. et al. TORCs: transducers of regulated CREB activity. Mol Cell 12, 413-423, doi:S1097276503003228 [pii] (2003).
59 Minokoshi, Y. et al. AMP-kinase regulates food intake by responding to hormonal and nutrient signals in the hypothalamus. Nature 428, 569-574, doi:10.1038/nature02440 (2004).
60 Tissenbaum, H. A. & Guarente, L. Increased dosage of a sir-2 gene extends lifespan in Caenorhabditis elegans. Nature 410, 227-230, doi:10.1038/35065638 35065638 [pii] (2001).

Having thus described in detail embodiments of the present invention, it is to be understood that the invention defined by the above paragraphs is not to be limited to particular details set forth in the above description as many apparent variations thereof are possible without departing from the spirit or scope of the present invention.

Each patent, patent application, and publication cited or described in the present application is hereby incorporated by reference in its entirety as if each individual patent, patent application, or publication was specifically and individually indicated to be incorporated by reference.

TABLE 2

| | | | | Sig | | | | | Log | |
|---|---|---|---|---|---|---|---|---|---|---|
| | | | | Lifespan | Day | Median | Median | % | Rank p | N |
| Strain | Description | Figure | RNAi | Diff? | Onset | Lifespan | Control | Increase | Value | (died/total) |
| N2 | Wild type | 1c | EV | | Hatch | 15 | 15 | 0 | | 88/100 |
| N2 | Wild type | 1c | CRTC | * | Hatch | 23 | 15 | 53.3 | <.0001 | 85/100 |
| N2 | Wild type | 1c | tax-6 | * | Hatch | 24 | 15 | 60 | <.0001 | 55/100 |
| N2 | Wild type | | EV | | Hatch | 16 | 16 | 0 | | 88/100 |
| N2 | Wild type | | CRTC | * | Hatch | 18 | 16 | 12.5 | 0.0002 | 86/100 |
| N2 | Wild type | | tax-6 | * | Hatch | 25 | 16 | 56.3 | <.0001 | 36/100 |
| N2 | Wild type | 1d | EV | | Hatch | 17 | 17 | | | 91/100 |
| AGD383 | AAK2 (aa3-321) | 1d | EV | * | Hatch | 20 | 17 | 17.6 | <0.0001 | |
| RB1667 | tax-6-null | 5a | EV | | Hatch | 19 | 19 | 0 | | 50/100 |
| RB1667 | tax-6-null | 5a | CRTC | Not Sig | Hatch | 18 | 19 | −5.3 | 0.2822 | 39/100 |
| RB1667 | tax-6 null | 5a | tax-6 | Not Sig | Hatch | 21 | 19 | 10.5 | 0.7363 | 45/100 |
| NL2099 | rrf-3 (pk1426) | | EV | | Hatch | 24 | 24 | 0 | | 60/100 |
| NL2099 | rrf-3 (pk1426) | | CRTC | * | Hatch | 30 | 24 | 25 | <.0001 | 71/100 |
| NL2099 | rrf-3 (pk1426) | | CRTC | * | Day 1 | 30 | 24 | 25 | <.0001 | 66/100 |
| NL2099 | rrf-3 (pk1426) | | CRH-1 | * | Hatch | 26 | 24 | 8.3 | 0.005 | 76/100 |
| NL2099 | rrf-3 (pk1426) | | CRH-1 | * | | 28 | 24 | 16.6 | <.0001 | 65/100 |
| NL2099 | rrf-3 (pk1426) | | EV | | HATCH | 22 | 22 | 0 | | 81/100 |
| NL2099 | rrf-3 (pk1426) | | CRTC | * | Day 1 | 27 | 22 | 22.7 | <.0001 | 76/100 |
| NL2099 | rrf-3 (pk1426) | | CRH-1 | * | Day 1 | 24 | 22 | 9.1 | 0.0027 | 76/100 |
| NL2099 | rrf-3 (pk1426) | | TAX-6 | * | Day 1 | 29 | 22 | 31.8 | <.0001 | 74/100 |
| NL2099 | rrf-3 (pk1426) | | W05G11.6 | * | Day 1 | 27 | 22 | 22.7 | <.0001 | 80/100 |
| NL2099 | rrf-3 (pk1426) | | R11A5.4 | * | Day 1 | 27 | 22 | 22.7 | <.0001 | 84/100 |
| NL2099 | rrf-3 (pk1426) | | GPI-1 | * | Day 1 | 27 | 22 | 22.7 | <.0001 | 74/100 |
| N2 | Wild type | 5b, 6d | EV | | Hatch | 20 | 20 | 0 | | 95/100 |
| N2 | Wild type | | CRTC | * | Hatch | 25 | 20 | 25 | <.0001 | 94/100 |
| N2 | Wild type | | CRH-1 | Not Sig | Hatch | 20 | 20 | 0 | 0.28 | 82/100 |
| N2 | Wild type | 5b | tax-6 | * | Hatch | 29 | 20 | 45 | <.0001 | 78/100 |
| N2 | Wild type | 6d | W05G11.6 | * | Hatch | 25 | 20 | 25 | <.0001 | 87/100 |
| N2 | Wild type | 6d | R11A5.4 | * | Hatch | 22 | 20 | 10 | <.0001 | 82/100 |
| N2 | Wild type | 6d | H04M03.1 | * | Hatch | 25 | 20 | 25 | <.0001 | 77/100 |
| N2 | Wild type | 6d | GPI-1 | * | Hatch | 25 | 20 | 25 | <.0001 | 68/100 |
| N2 | Wild type | 6d | pyc-1 | * | Hatch | 22 | 20 | 10 | 0.01 | 94/100 |
| AGD466 | CRTC-1 | 5b | EV | | Hatch | 18 | 20 | −10 | 0.07 | 37/60 |
| | (S76A, (s179A) | | | | | | | | | |
| AGD466 | CRTC-1 | 5b | Tax-6 | Not Sig | Hatch | 20 | 18 | 11.1 | 0.74 | 30/60 |
| | (S76A, (s179A) | | | | | | | | | |
| N2 | Wild type | | EV | | Hatch | 20 | 20 | 0 | | 94/100 |
| N2 | Wild type | | CRH-1 | Not Sig | Hatch | 18 | 20 | −10 | 0.74 | 81/100 |
| N2 | Wild type | | CRH-1 | Not Sig | Day 1 | 21 | 20 | 5 | 0.11 | 72/100 |
| NL2099 | rrf-3 (pk1426) | 6c | EV | | Hatch | 22 | 22 | 0 | | 80/100 |
| NL2099 | rrf-3 (pk1426) | 6c | CRH-1 | * | Hatch | 23 | 22 | 4.5 | 0.003 | 65/100 |
| NL2099 | rrf-3 (pk1426) | 6c | CRH-1 | * | Day 1 | 24 | 22 | 9.1 | 0.0001 | 81/100 |
| AGD418 | CRTC-1 | 5c | EV | | Hatch | 23 | 23 | 0 | | 79/100 |
| AGD418 | CRTC-1 | 5c | tax-6 | * | Hatch | 27 | 23 | 17.4 | <.0001 | 73/100 |
| AGD466 | CRTC-1 | 5c | EV | | Hatch | 20 | 23 | −13.0 | 0.48 | 71/100 |
| | (S76A, (s179A) | | | | | | | | | |
| AGD466 | CRTC-1 | 5c | tax-6 | Not Sig | Hatch | 21.5 | 20 | 7.5 | 0.57 | 60/100 |
| | (S76A, (s179A) | | | | | | | | | |
| N2 | Wild type | 11 | EV | | Hatch | 19 | 19 | 0 | | 95/100 |
| N2 | Wild type | 11 | tax-6 | * | Hatch | 26 | 19 | 36.8 | <.0001 | 19/100 |
| N2 | Wild type | 11 | tax-6 | * | L4 | 23 | 19 | 21.1 | <.0001 | 90/100 |
| NL2099 | rrf-3 (pk1426) | 11 | EV | | Hatch | 22 | 22 | 0 | | 76/100 |
| NL2099 | rrf-3 (pk1426) | 11 | tax-6 | * | Hatch | 28 | 22 | 27.3 | <.0001 | 86/100 |
| NL2099 | rrf-3 (pk1426) | 11 | tax-6 | * | L4 | 28 | 22 | 27.3 | <.0001 | 83/100 |
| AGD466 | CRTC-1 | 11 | EV | | Hatch | 20 | 20 | 0 | | 82/100 |
| | (S76A, (s179A) | | | | | | | | | |
| AGD466 | CRTC-1 | 11 | tax-6 | | Hatch | 20 | 20 | 0 | 0.38 | 40/100 |
| | (S76A, (s179A) | | | | | | | | | |
| AGD466 | CRTC-1 | 11 | tax-6 | | L4 | 20 | 20 | 0 | 0.26 | 79/100 |
| | (S76A, (s179A) | | | | | | | | | |

TABLE 3

| | Mammalian Gene | C. elegans ortholog |
|---|---|---|
| LKB complex | LKB1 | par-4 |
| | STRADa | strd-1 |
| | STRADb | gck-3 |
| | MO25a | Y53C12A.1 |
| | MO25b | R02E12 |
| AMPK family kinases | AMPK a1 | aak-1 |
| | AMPK a2 | aak-2 |
| | SIK | kin-29 |
| | MARK2 | F23C8.8 |
| | MARK3 | par-1 |
| | Nuak1/2 | B0496.3 |
| | SNRK | ZK524.4 |
| | SAD A/B | sad-1 |

1. A recombinant C. elegans that expresses a detectable marker operably linked to a CRTC protein in intestinal cells.

2. The recombinant C. elegans of claim 1, wherein the detectable marker is further defined as a marker that can be visually detected.

3. The recombinant C. elegans of claim 1, wherein the detectable marker is further defined as a marker that can be spectroscopically detected.

4. The recombinant C. elegans of claim 1, wherein the detectable marker is a green fluorescent protein.

5. The recombinant C. elegans of claim 1, wherein the detectable marker is a yellow fluorescent protein.

6. The recombinant C. elegans of claim 1, wherein the detectable marker is a blue fluorescent protein.

7. The recombinant C. elegans of claim 1, wherein the detectable marker is a red fluorescent protein.

8. A method of screening test compounds to determine whether such compounds affect the activity of an AMP-activated kinase or an LKB1 kinase, said method comprising determining the effect of the test compound on the localization of a CRTC protein in the recombinant C. elegans of claim 1.

9. A method of increasing lifespan in an organism, comprising modulating expression or location of a CRTC protein.

10. A method of increasing lifespan in an organism, comprising modulating expression of a CREB protein.

11. (canceled)

12. (canceled)

13. A composition comprising isolated nucleic acid encoding a C. elegans CRTC protein.

14. The composition of claim 13 wherein the CRTC protein is CRTC-I.

15. The method of claim 9, comprising modulating phosphorylation of the CRTC protein.

16. The method of claim 9, wherein the expression of the CRTC protein is reduced.

17. The method of claim 15, comprising inhibiting calcineurin activity.

18. The method of claim 15, comprising enhancing AMPK activity.

19. A method of screening test compounds to determine whether such compounds affect the activity of calcineurin or enhance longevity, said method comprising determining the effect of the test compound on the localization of a CRTC protein.

20. (canceled)


SEQUENCE LISTING (10 sequences)

SEQ ID NO: 1 - protein, 473 aa, Caenorhabditis elegans
MSNSNTPRKFSEKIAILERKQNEENTTFEDIMRQVQSITHHPTDSSGSSTATAPMPIPQQGLLPPQQPWGHNLGGSLPNVHQMPSYSPPQWPPNWQHEIQHRPIQGHRSRSPEDHMIGSASGSPSHHYHPYGMRSNGLSRSPDRTPPQHPQYTPYGPPYNQPGQLVPPESWNQINRARSDPAIHNMGGMVPMHPHHHQQQQMAFHQMPHYLQNSMPGPSGMMAPNSQSQQHSPQLTPQGSQQGSPVQMHHQIPPPLQMGNNQQMGGGNNGMSPYHNGSPLHSPMDSPHASTLMLDGSGTPSPYMEVSPPGMHDFNQDAGSLPNLQNVQQQQQQQQEIYTNGGAPGGGYYHAPIGPRHSTGACGPRLVPGPALTPESQSAPTSPHNQLDPNQPPMWPTRTFSNSPEALDIPKLTITNAEGAPGHHVDSYNDFNDLGLDSLDSVLCNGAPPQTISNHQTPNNSFHDPGGTQMLQN

SEQ ID NO: 2 - protein, 13 aa, Caenorhabditis elegans
LQSPNHMMTPMYG

SEQ ID NO: 3 - protein, 16 aa, Caenorhabditis elegans
EALDIPKLTITNAEGA

SEQ ID NO: 4 - protein, 16 aa, Caenorhabditis elegans
EALDIAKATAANAEGA

SEQ ID NO: 5 - protein, 6 aa, Caenorhabditis elegans
PKLTIT

SEQ ID NO: 6 - protein, 5 aa, Caenorhabditis elegans
GGSLP

SEQ ID NO: 7 - protein, 6 aa, Caenorhabditis elegans
RARSDP

SEQ ID NO: 8 - protein, 184 aa, Caenorhabditis elegans
SNSNTPRKFSEKIAILERKQNEENTTFEDIMRQVQSITHHPTDSSGSSTATAPMPIPQQGLLPPQQPWGHNLGGSLPNVHQMPSYSPPQWPPNWQHEIQHRPIQGHRSRSPEDHMIGSASGSPSHHYHPYGMRSNGLSRSPDRTPPQHPQYTPYGPPYNQPGQLVPPESWNQINRARSDPAIHN

SEQ ID NO: 9 - protein, 159 aa, Homo sapiens
ATASASNPRKFSEKIALQKQRQAEETAAFEEVMMDIGSTRLQAQKLRLAYTRSSHYGGSLPNVNQIGSGLAEFQSPLHSPLDSSRSTRHHGLVERVQRDPRRMVSPLRRYTRHIDSSPYSPAYLSPPPESSWRRTMAWGNFPAEKGQLFRLPSALNRTS

SEQ ID NO: 10 - DNA, 1460 bp, Caenorhabditis elegans
atgtcaaattcgaatacaccacgcaagttcagtgagaagattgcaatactcgagaggaaacagaatgaggagaatacaacgtttgaggatattatgcgacaggtgcaatcaatcacccatcatccgacggattcatcaggatcatcgacggcaactgcaccaatgcccattccgcaacagggtctccttccacctcaacaaccatggggtcacaatctcggaggatccctgccgaacgtccatcaaatgccatcctactctccaccacaatggccaccaaattggcagcacgagattcaacacagacctatccaaggacatcgaagtcgatcaccagaggatcacatgatcggaagtgcatctggatcaccatcacatcactatcatccgtatggaatgagatcgaatggattatcacgatcgcccgatcgaacgcctccccaacacccgcagtatacgccctatggacccccgtataaccaacctggacagcttgtgccgcccgagtcgtggaatcaaatcaatcgtgcacgttcggatcctgcaattcataacatgggcggtatggttccaatgcatccacatcaccatcaacaacaacaaatggcatttcatcaaatgcctcattatcttcaaaattcgatgcctggaccatctggaatgatggctccgaacagtcaatcacaacaacactcacctcagttaacacctcaaggatcgcagcaaggatcaccggttcagatgcatcatcagatacctccaccattacaaatgggcaataaccaacaatgggcggaggcaataatggaatgtcaccgttacaatcacctaatcatatgatgacacctatgtatgggtatcataatggctcaccactccattcaccgatggattcaccccatgcctcaacattaatgctcgatggatcagggacaccctctccgtatatggaagtaagtcctcccggaatgcatgatttcaatcaagatgccggaagtttacccaatcttcaaaatgtgcaacagcagcagcaacagcaacaggaaatttatacaaacgggggagcccctggtggtggatactatcatgctccaattggaccaagacatagtacaggtgcatgtggtccgaggctagtacccggtccagcgttgacacccgaatcacagtcggcgccgacgtcaccgcataatcaattggatccgaatcaaccgcccatgtggccgacgagaacattctccaattcaccggaggcattggatattccgaaattgacaattacaaatgctgaaggtgcacctgggcatcatgttgattcgtataatgattttaatgatcttggcctcgactcactggacagcgtgttatgtaacggagcgccgccacagacaatatccaaccaccaaacacccaacaactcattccatgatccgggcggtacacaaatgctccaaaattag